(19) United States
(12) Patent Application Publication
(10) Pub. No.: US 2011/0020336 A1
(43) Pub. Date: Jan. 27, 2011

(54) Combination Of A Beta-Glucan And An EGF Receptor Antagonist For The Treatment Of Cancer And Infection

(76) Inventor: Steven J. Karel, Mendota, MN (US)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/896,692

(22) Filed: Oct. 1, 2010

Related U.S. Application Data
(63) Continuation of application No. 11/919,927, filed on Feb. 27, 2009.
(60) Provisional application No. 60/677,212, filed on May 3, 2005.

(51) Int. Cl. A61K 39/395; A61K 31/715

(52) U.S. Cl. 424/133.1; 424/130.1; 424/145.1; 514/54

(57) ABSTRACT

The present invention encompasses a therapeutic combination composition of a β-glucan EGF receptor antagonist. This therapeutic combination composition is useful for the treatment of diseases including proliferative disorders and immune dysfunction.

### FIELD OF THE INVENTION

The present invention relates to methods and compositions for treating proliferative disorders or immune dysfunctions. More specifically, the present invention relates to compositions of β-glucan and EGF receptor antagonists for treating cancer and infections.

### BACKGROUND

In the early 1960's, zymosan, a crude insoluble yeast extract prepared by boiling yeast before and after trypsin treatment, was noted to produce marked hyperplasia and functional stimulation of the reticuloendothelial system in rodents. In animal studies, zymosan preparations were shown to inactivate complement component C3, to enhance antibody formation, to promote survival following irradiation, to increase resistance to bacterial infections, to inhibit tumor development, to promote graft rejection, and to inhibit dietary-induced hypercholesterolemia and cholesterosis. Zymosan was shown to consist of polysaccharides, proteins, fats; and inorganic elements; however, subsequent studies identified the active components of the yeast cell wall as a pure polysaccharide, specifically β-glucan. Repetition of biological assays with β-glucan indicated that most of the above functional activities identified with zymosan were retained by the purified β-glucan preparation.

The properties of β-glucan are quite similar to those of endotoxin in increasing nonspecific immunity and resistance to infection. The activities of β-glucan as an immune adjuvant and hemopoietic stimulator compare to those of more complex biological response modifiers (BRMs), such as bacillus Calmette-Guerin (BCG) and Corynebacterium parvum. The functional activities of yeast β-glucan are also comparable to those structurally similar carbohydrate polymers isolated from fungi and plants. These higher molecular-weight β-(1-3)-D-glucans such as schizophyllan, lentinan, krestin, grifolan, and pachyman exhibit similar immunomodulatory activities. A common mechanism shared by all these β-glucan preparations is their stimulation of cytokines such as interleukin-1 and (IL-1). (TNF) Lentinan has been extensively investigated for its antitumor properties, both in animal models at 1 mg/kg for 10 days and in clinical trials since the late 1970s in Japan for advanced or recurrent malignant lymphoma and colorectal, mammary, lung and gastric cancers. In cancer chemotherapy, lentinan has been administered at 0.5-5 mg/day, I.M. or I.V., two or three times per week alone, or in combination with antineoplastic drugs. In addition to the activities ascribed to yeast glucans, studies suggest lentinan acts as a T-cell immunopotentiator, inducing cytotoxic activities, including production of IL-1, colony-stimulating factor (CSP) and IL-3. (Chihara et al., 1989, Int. J. Immunotherapy, 4:145-154; Hamuro and Chihara, In Lentinan, An Immunopotentiator).

Cetuximab (ERBITUX™) is a recombinant, human/mouse chimeric monoclonal antibody that binds specifically to the extracellular domain of the human epidermal growth factor receptor (EGFR). Cetuximab is composed of the Fv regions of a murine anti-EGFR antibody with human IgG1 heavy and kappa light chain constant regions and has an approximate molecular weight of 152 kDa. Cetuximab is produced in mammalian (murine myeloma) cell culture.

Cetuximab binds specifically to the epidermal growth factor receptor (EGFR, HER1, c-ErbB-1) on both normal and tumor cells, and competitively inhibits the binding of epidermal growth factor (EGF) and other ligands, such as transforming growth factor-alpha. Binding of Cetuximab to the EGFR blocks phosphorylation and activation of receptor-associated kinases, resulting in inhibition of cell growth, induction of apoptosis, and decreased matrix metalloproteinase and vascular endothelial growth factor production. The EGFR is a transmembrane glycoprotein that is a member of a subfamily of type I receptor tyrosine kinases including EGFR (HER1), HER2, HER3, and HER4. The EGFR is constitutively expressed in many normal epithelial tissues, including the skin and hair follicle. Over-expression of EGFR is also detected in many human cancers including those of the colon and rectum.

In vitro assays and in vivo animal studies have shown that Cetuximab inhibits the growth and survival of tumor cells that over-express the EGFR. The addition of Cetuximab to irinotecan or irinotecan plus 5-fluorouracil in animal studies resulted in an increase in anti-tumor effects compared to chemotherapy alone.

The composition of the present invention comprises both β-glucan and an EGF receptor antagonist used for treatment of immune dysfunction and proliferative disorders.

### SUMMARY OF THE INVENTION

The invention encompasses a combination composition including a β-glucan and an EGF receptor antagonist, methods of treating diseases, such as proliferative disorders and immune dysfunctions with the composition, kits comprising the β-glucan and an EGF receptor antagonist, and pharmaceutical compositions for the treatment of proliferative disorders and immune dysfunctions with β-glucans and an EGF receptor antagonists.

The invention provides a composition including a β-glucan and an EGF receptor antagonist. In one embodiment of the composition of the invention, the β-glucan forms a triple helix. In one aspect of this embodiment, the triple helix β-glucan forms a higher order aggregate. Optionally, the higher order aggregate has an aggregate number selected from the group consisting of 5, 6, 7, 8, 9, 10, 11, 12, 13, 14, 15, 16, 17, 18, 19 and 20.

In another embodiment of the composition of the invention, the EGF receptor antagonist is an antibody. In one aspect of this embodiment, the antibody is polyclonal. In another aspect of this embodiment, the antibody is monoclonal. Optionally, the monoclonal antibody is antibody 108 or antibody 96 disclosed in U.S. Pat. No. 6,217,866, incorporated herein by reference.

In another embodiment of the composition of the invention, the EGF receptor is a chimeric antibody. In one aspect of this embodiment, the chimeric antibody is Cetuximab.

In another embodiment of the composition of the invention, the composition further includes an anti-cancer drug. In one aspect of this embodiment, the anti-cancer drug is ironotecan, doxorubicin or cisplatin.

In another embodiment of the composition of the invention, the composition is administered to a subject. In one aspect of this embodiment, the subject is a mammal. Optionally, the mammal is a human. In another aspect of this embodiment, the β-glucan is administered to the subject at a dose from about 0.1 to about 2.5 mg/kg/day. In another aspect of this embodiment, the EGF receptor antagonist is administered to the subject at a dose from about 125 to about 800 mg/m² per week. In another aspect of this embodiment, the β-glucan and EGF receptor antagonist are both administered in one infusion about once a week.

The invention also provides a method of treating a proliferative disorder in a subject, the method comprising administering to the subject an effective amount of a β-glucan and an effective amount of an EGF receptor antagonist, thereby treating the proliferative disorder in the subject. In one embodiment of the method of treating a proliferative disorder in a subject, the proliferative disorder is cancer. In one aspect of this embodiment, the cancer is ovarian cancer, breast cancer, prostate cancer, colon cancer, pancreatic cancer, multiple myeloma, malignant melanoma or non-melanoma skin cancer.

In another embodiment of the method of treating a proliferative disorder in a subject, the β-glucan forms a triple helix. In one aspect of this embodiment, the triple helix β-glucan forms a higher order aggregate. Optionally, the higher order aggregate has an aggregate number selected from the group consisting of 5, 6, 7, 8, 9, 10, 11, 12, 13, 14, 15, 16, 17, 18, 19 and 20.

In another embodiment of the method of treating a proliferative disorder in a subject, the EGF receptor antagonist is an antibody. In one aspect of this embodiment, the antibody is polyclonal. In another aspect of this embodiment, the antibody is monoclonal. Optionally, the monoclonal antibody is antibody 108 or antibody 96, described above.

In another embodiment of the method of treating a proliferative disorder in a subject, the EGF receptor is a chimeric antibody. In one aspect of this embodiment, the chimeric antibody is Cetuximab.

In another embodiment of the method of treating a proliferative disorder in a subject, the method further includes the step of administering an anti-cancer drug. In one aspect of this embodiment, the anti-cancer drug is ironotecan, doxorubicin or cisplatin.

In another embodiment of the method of treating a proliferative disorder in a subject, the subject is a mammal. In another aspect of this embodiment, the mammal is a human.

In another embodiment of the method of treating a proliferative disorder in a subject, the β-glucan is administered to the subject at a dose from about 0.1 to about 2.5 mg/kg/day.

In another embodiment of the method of treating a proliferative disorder in a subject, the EGF receptor antagonist is administered to the subject at a dose from about 125 to about 800 mg/m² per week.

In another embodiment of the method of treating a proliferative disorder in a subject, the β-glucan and EGF receptor antagonist are both administered in one infusion about once a week.

38. A method of treating an immune dysfunction in a subject, the method comprising administering to the subject an effective amount of a β-glucan and an effective amount of an EGF receptor antagonist, thereby treating the immune dysfunction in the subject.

The invention also provides a method of treating an immune dysfunction in a subject, the method comprising administering to the subject an effective amount of a β-glucan and an effective amount of an EGF receptor antagonist, thereby treating the immune dysfunction in the subject. In one embodiment of the method of treating an immune dysfunction in a subject, the immune dysfunction is infection.

In another embodiment of the method of treating an immune dysfunction in a subject, the β-glucan forms a triple helix. In one aspect of this embodiment, the triple helix β-glucan forms a higher order aggregate. Optionally, the higher order aggregate has an aggregate number selected from the group consisting of 5, 6, 7, 8, 9, 10, 11, 12, 13, 14, 15, 16, 17, 18, 19 and 20.

In another embodiment of the method of treating an immune dysfunction in a subject, the EGF receptor antagonist is an antibody. In one aspect of this embodiment, the antibody is polyclonal. In another aspect of this embodiment, the antibody is monoclonal. Optionally, the monoclonal antibody is antibody 108 or antibody 96, described above.

In another embodiment of the method of treating an immune dysfunction in a subject, the EGF receptor is a chimeric antibody. In one aspect of this embodiment, the chimeric antibody is Cetuximab.

In another embodiment of the method of treating an immune dysfunction in a subject, the subject is a mammal. In another aspect of this embodiment, the mammal is a human.

In another embodiment of the method of treating an immune dysfunction in a subject, the β-glucan is administered to the subject at a dose from about 0.1 to about 2.5 mg/kg/day.

In another embodiment of the method of treating an immune dysfunction in a subject, the EGF receptor antagonist is administered to the subject at a dose from about 125 to about 800 mg/m² per week.

In another embodiment of the method of treating an immune dysfunction in a subject, the β-glucan and EGF receptor antagonist are both administered in one infusion about once a week.

The invention also provides a kit containing a therapeutic dose of a β-glucan and a therapeutic dose of an EGF receptor antagonist either in the same or separate packaging, and instructions for its use. In one embodiment of the kit the β-glucan forms a triple helix. In one aspect of this embodiment the triple helix β-glucan forms a higher order aggregate. Optionally, the higher order aggregate has an aggregate number selected from the group consisting of 5, 6, 7, 8, 9, 10, 11, 12, 13, 14, 15, 16, 17, 18, 19 and 20.

In another embodiment of the kit, the EGF receptor antagonist is an antibody. In one aspect of this embodiment, the antibody is polyclonal. In another aspect of this embodiment, the antibody is monoclonal. Optionally, the monoclonal antibody is antibody 108 or antibody 96, described above.

In another embodiment of the kit, the EGF receptor is a chimeric antibody. In one aspect of this embodiment, the chimeric antibody is Cetuximab.

In another embodiment of the kit, the β-glucan is administered to the subject at a dose from about 0.1 to about 2.5 mg/kg/day.

In another embodiment of the kit, the EGF receptor antagonist is administered to the subject at a dose from about 125 to about 800 mg/m² per week.

The invention also provides a pharmaceutical composition comprising a β-glucan and a EGF receptor antagonist in an effective amount to treat cancer. In one embodiment of the pharmaceutical composition to treat cancer, the β-glucan is a triple helical β-glucan and the EGF receptor antagonist is Cetuximab.

In another embodiment of the pharmaceutical composition to treat cancer, the composition also includes an anti-cancer drug in an effective amount to treat cancer. In one aspect of this embodiment the anti-cancer drug is ironotecan, doxorubicin or cisplatin.

The invention also provides a pharmaceutical composition comprising a β-glucan and a EGF receptor antagonist in an effective amount to treat infection. In one embodiment of the pharmaceutical composition to treat cancer, the β-glucan is a triple helical β-glucan and the EGF receptor antagonist is Cetuximab.

### DETAILED DESCRIPTION OF THE INVENTION

The invention encompasses a composition including both a β-glucan and an EGF receptor antagonist. Because of the proven ability of β-glucans to treat immune dysfunction including infections and other immune problems associated with chemotherapy, radiation treatment and other cancer treatments, and EGF receptor antagonists have been shown to be effective in the treatment of cancer, the two active ingredients will work additively or synergistically in the treatment of proliferative disorders or immune dysfunction. Further, other pharmaceuticals are used with the composition of the invention. For example, other anti-cancer drugs are combined with a β-glucan and an EGF receptor antagonist for the treatment of a proliferative disorder. Also, more than one β-glucan or EGF receptor antagonist are used in the same composition. For example, a triple helical β-glucan is combined with a β-glucan with an aggregate number of 7 (meaning that the aggregate contains 7 β-glucan chains) which is further combined with Cetuximab.

The β-glucans used in the invention include PGG (poly-(1-6)-β-D-glucopyranosyl-(1-3)-β-D-glucopyranose), neutral soluble β-glucan, triple helical β-glucan (BETAFECTIN™), and β-glucans of various aggregate numbers. The above mentioned species of β-glucans are administered separately or in various combinations. EGF receptor antagonists used in the composition of the invention include polyclonal and monoclonal antibodies, recombinant human/mouse chimeric monoclonal antibody (Cetuximab), antibody fragments, other proteins and small molecules that bind specifically to the extracellular domain of the human epidermal growth factor receptor. The above mentioned species of EGF receptor antagonists are administered separately or in various combinations.

The invention also encompasses a method of treating a proliferative disorder in a mammal by administering to the mammal a composition including both a β-glucan and an EGF receptor antagonist. The method may further comprise the administration of other anti-cancer drugs with the β-glucan and an EGF receptor antagonist. The invention also encompasses a method of treating an immune dysfunction in a mammal by administering to the mammal a composition including both a β-glucan and an EGF receptor antagonist.

The compositions may, if desired, be presented in a pack or dispenser device and/or a kit which may contain one or more unit dosage forms containing the active ingredients. The pack may for example comprise metal or plastic foil, such as a blister pack. The pack or dispenser device may be accompanied by instructions for administration.

β-Glucans

The β-glucan preparations of this invention are prepared from insoluble glucan particles. Manners et al., Biol. J., 135:19-30, (1973). β-glucan is also referred to herein as PGG (poly-(1-6)-β-D-glucopyranosyl-(1-3)-β-D-glucopyranose). A β-glucan polysaccharide can exist in at least four distinct conformations: single disordered chains, single helix, single triple helix and triple helix aggregates. The terms “neutral soluble β-glucan” and “neutral soluble glucan” are intended to mean an aqueous soluble β-glucan having a unique triple helical conformation that results from the denaturation and re-annealing of aqueous soluble glucan. Single chains are also isolated and used, i.e., not substantially interacting with another chain. Three single helix chains can combine to form a triple helix structure which is held together by interchain hydrogen bonding. Two or more β-glucan triple helices can join together to form a triple helix aggregate. Preparations of the β-glucan can comprise one or more of these forms, depending upon such conditions as pH and temperature.

Glucan particles which are particularly useful as starting materials in the present invention are whole glucan particles described by Jamas et al., in U.S. Pat. Nos. 4,810,646, 4,992,540, 5,082,936 and 5,028,703, the teaching of all are hereby incorporated herein by reference. The source of the whole glucan particles can be the broad spectrum of glucan-containing fungal organisms which contain β-glucans in their cell walls. Whole glucan particles obtained from the strain Saccharomyces cerevisiae R4 (NRRL Y-15903; deposit made in connection with U.S. Pat. No. 4,810,646) and R4 Ad (ATCC No. 74181) are particularly useful. The structurally modified glucans hereinafter referred to as “modified glucans” derived from S. cerevisiae R4 are potent immune system activators, as described in Jamas et al. in U.S. Pat. No. 5,504,079, the teachings of which are hereby incorporated herein by reference.

The whole glucan particles utilized in this present invention can be in the form of a dried powder, as described by Jamas et al., in U.S. Pat. Nos. 4,810,646, 4,992,540, 5,082,936 and 5,028,703. For the purpose of this present invention it is not necessary to conduct the final organic extraction and wash steps described by Jamas et al.

The soluble glucans produced by the method shown in Example 4, below, are branched polymers of glucose, referred to as PGG, containing β(1-3) and β(1-6) linkages in varying ratios depending on the organism and processing conditions employed. The PGG glucan preparations contain neutral glucans, which have not been modified by substitution with functional (e.g., charged) groups or other covalent attachments. The biological activity of PGG glucan can be controlled by varying the average molecular weight and the ratio of β(1-6) to β(1-3) linkages of the glucan molecules, as described by Jamas et al. in U.S. Pat. Nos. 4,810,646, 4,992,540, 5,082,936 and 5,028,703. The average molecular weight of soluble glucans produced by the present method is generally from about 10,000 to about 500,000 daltons, preferably from about 30,000 to about 50,000.

For purposes of the present invention, the term “soluble” as used herein to describe glucans obtained by the present process, means a visually clear solution can be formed in an aqueous medium such as water, PBS, isotonic saline, or a dextrose solution having a neutral pH (e.g., about pH 5 to about 7.5), at room temperature (about 20-25° C.) and at a concentration of up to about 10 mg/ml. The term “aqueous medium” refers to water and water-rich phases, particularly to pharmaceutically acceptable aqueous liquids, including PBS, saline and dextrose solutions.

Neutral Soluble β-Glucan

Neutral soluble β-glucan (also referred to as BETAFECTIN™) has been shown to increase the number of neutrophils and monocytes as well as their direct infection fighting activity (phagocytosis and microbial killing). However, the neutral soluble β-glucan does not stimulate the production of biochemical mediators, such as IL-1, TNF and leukotrienes, that can cause detrimental side effects such as high fever, inflammation, wasting disease and organ failure. These advantageous properties make neutral soluble glucan preparations useful in the prevention and treatment of infection because they selectively activate only those components of the immune system responsible for the initial response to infection, without stimulating the release of certain biochemical mediators that can cause adverse side effects. The solution containing the neutral soluble β-glucan also lacks the toxicity common to many immunomodulators.

The neutral soluble β-glucans of this invention are composed of glucose monomers organized as a β(1-3) linked glucopyranose backbone with periodic branching via β(1-6) glycosidic linkages. The neutral soluble glucan preparations contain glucans, which have not been substantially modified by substitution with functional (e.g., charged) groups or other covalent attachments. The general structure of the neutral soluble glucan is shown in FIG. 1 of U.S. Pat. No. 5,488,040, incorporated herein, by reference. One biologically active preparation of this invention is a conformationally purified form of β-glucan produced by dissociating the native glucan conformations and re-annealing and purifying the resulting unique triple helical conformation. The unique conformation of the neutral soluble glucan contributes to the glucan's ability to selectively activate the immune system without stimulating the production of detrimental biochemical mediators. Methods of making neutral soluble β-glucans are shown in Example 5.

The neutral soluble glucan preparations of this invention are prepared from insoluble glucan particles, preferably derived from yeast organisms as described herein. Other strains of yeast that can be used include Saccharomyces delbrueckii, Saccharomyces rosei, Saccharomyces microellipsodes, Saccharomyces carlsbergensis, Schizosaccharomyces pombe, Kluyveromyces lactis, Kluyveromyces fragilis, Kluyveromyces polysporus, Candida albicans, Candida cloacae, Candida tropicalis, Candida utilis, Hansenula winged, Hansenula arni, Hansenula izenricii, Hansenula americana. A procedure for extraction of whole glucan particles is also described herein.

β-Glucan in an Aggregate Conformation

The term “single triple helix”, as used herein, refers to a β-glucan conformation wherein three single chains are joined together to form a triple helix structure. In this conformation, there is no higher ordering of these triple helices, that is, there is no substantial aggregation of triple helices.

The term “triple helix aggregate”, as used herein, refers to a β-glucan conformation in which two or more triple helices are joined together via non-covalent interactions.

The “molecular weight” of a β-glucan composition, as the term is used herein, is the mass average molar mass of the collection of polymer molecules within the composition. The characterization of a collection of polymer molecules in terms of polymer mass average molar mass is well known in the art of polymer science.

The “aggregate number” of a β-glucan conformation is the number of single chains which are joined together in that conformation. The aggregate number of a single helix is 1, the aggregate number of a single triple helix is 3, and the aggregate number of a triple helix aggregate is greater than 3. For example, a triple helix aggregate consisting of two triple helices joined together has an aggregate number of 6.

The aggregate number of a β-glucan sample under a specified set of conditions can be determined by determining the average molecular weight of the polymer under those conditions. The β-glucan is then denatured, that is, subjected to conditions which separate any aggregates into their component single polymer chains. The average molecular weight of the denatured polymer is then determined. The ratio of the molecular weights of the aggregated and denatured forms of the polymer is the aggregate number. A typical β-glucan composition includes molecules having a range of chain lengths, conformations and molecular weights. Thus, the measured aggregate number of a β-glucan composition is the mass average aggregate number across the entire range of β-glucan molecules within the composition. It is to be understood that any reference herein to the aggregate number of a β-glucan composition refers to the mass average aggregate number of the composition under the specified conditions. The aggregate number of a composition indicates which conformation is predominant within the composition. For example, a measured aggregate number of about 6 or more is characteristic of a composition in which the β-glucan is substantially in the triple helix aggregate conformation.

The conformation of a PGG-glucan preparation is temperature dependent. For example, an aqueous PGG-glucan solution prepared according to the method disclosed in U.S. Pat. No. 5,622,939, incorporated herein by reference, elutes from a gel permeation chromatography column (GPC, also referred to as size exclusion chromatography) at 25° C. as a single symmetric peak. When the elution is conducted at 37° C. however, two distinct peaks are observed, denoted Fraction A, which elutes first, and Fraction C, which elutes last.

The molecular weights of fractions A and C were determined at 25° C. at both pH 7 and pH 13, and at 37° C. at pH 7. At pH 13, PGG-glucan is in an unaggregated or single chain conformation. Thus, at a given temperature the ratio of the molecular weights determined at pH 7 and pH 13 is the aggregate number at pH 7 at that temperature.

At pH 7 and 25° C., Fraction A had a molecular weight of 238,000 and an aggregate number of 15.0. Upon increasing the temperature to 37° C., the molecular weight of Fraction A decreased to 164,000 and the aggregate number decreased to 10.3. At 75° C. the molecular weight of this fraction was 52,600 with an aggregate number of 3.3. The temperature dependence of molecular weight and aggregate number was more pronounced for Fraction C. At pH 7.0 and 25° C., Fraction C had a molecular weight of 71,500 and an aggregate number of 6.0. At 37° C., the molecular weight of Fraction C was 32,000 and the aggregate number was 2.7. At 75° C., the molecular weight of this fraction was 17,200 and the aggregate number was 1.4.

The results of this study indicate that at 25° C. and pH 7, both Fraction A and Fraction C exist predominantly in a triple helix aggregate conformation. When the temperature is increased to 37° C., Fraction A remains predominantly in a triple helix aggregate conformation, while Fraction C is primarily in a single triple helix conformation. At 75° C., Fraction A remains predominantly in a single triple helix conformation, while Fraction C is primarily in a single chain random coil conformation.

In another series of experiments, the original PGG-glucan preparation described above was subjected to preparative scale GPC at 25° C., resulting in a single broad elution band. Portions from the leading and trailing edges and the center of this band were collected to provide, in order of elution, Fractions 1, 2 and 3. The average molecular weight of each fraction was determined at both pH 7 and pH 13. The results showed that both molecular weight and aggregate number decreased with increasing elution time. The molecular weights determined at 25° C. ranged from 244,100 for Fraction 1, 156,600 for Fraction 2, and 104,300 for Fraction 3. The aggregate numbers determined at 25° C. were 11.3 for Fraction 1, 8.6 for Fraction 2 and 7.7 for Fraction 3.

The average molecular weight and aggregate number of each fraction were temperature dependent. For each fraction, both average molecular weight and aggregate number decreased upon warming from 25° C. to 37° C. The molecular weights (aggregate numbers) determined at 37° C. were 164,100 (7.6) for Fraction 1, 109,100 (6.0) for Fraction 2, and 51,760 (3.8) for Fraction 3.

These results indicate that in each fraction the PGG-glucan is predominantly in a triple helix aggregate conformation at 25° C. At 37° C., however, Fractions 1 and 2 remain predominantly in a triple helix aggregate conformation, while Fraction 3, however, is primarily in a single triple helix conformation.

The aggregation state of another β-glucan, known as scleroglucan, was also examined. Scleroglucan is a β-glucan polymer which is substantially more branched than PGG-glucan. Based upon the molecular weights of a scleroglucan sample at 25° C. at pH 7 and pH 13 and at 37° C. and pH 7, the aggregate number of this sample was determined to be about 3 at both temperatures. Thus while PGG-glucan exists in a triple helix aggregate conformation at 25° C. and pH 7, under these conditions scleroglucan exists primarily in a single triple helix conformation.

The differences in the conformations of scleroglucan and PGG-glucan can be ascribed to structural differences between the two β-glucans. As the primary structural difference is the extent of branching, this suggests that scleroglucan is too highly branched to form triple helix aggregates under these conditions. This indicates that a β-glucan which forms triple helix aggregates at physiological temperature and pH can be formed by debranching a highly branched β-glucan such as scleroglucan.

The present invention also provides a soluble β-glucan composition which is substantially in a triple helix aggregate conformation under physiological conditions.

The term “physiological conditions”, as used herein, refers to physiological pH, about pH 7, and physiological temperature, about 37° C. In a preferred embodiment, under physiological conditions the β-glucan composition consists essentially of β-glucan chains in one or more triple helix aggregate conformations.

As used herein, a soluble β-glucan composition is “substantially in a triple helix conformation” if greater that about 50% by weight of the composition is in a triple helix aggregate conformation under physiological conditions. Preferably, greater than about 60%, and more preferably, greater than about 70% by weight of the composition is in a triple helix aggregate conformation under physiological conditions. In one embodiment, the soluble β-glucan composition of the invention is characterized by an aggregate number under physiological conditions of greater than about 6. Preferably, the aggregate number of the β-glucan composition under physiological conditions is at least about 7, and, more preferably, at least about 8. In the most preferred embodiment, the aggregate number of the β-glucan composition under physiological conditions is at least about 9.

In another embodiment, the present invention provides a method of preparing a soluble β-glucan composition having an aggregate number greater than that of a starting soluble β-glucan composition. The method comprises separating a high molecular weight portion from a starting soluble β-glucan composition. The high molecular weight portion is enriched in the triple helix aggregate conformation compared to the starting composition. The starting composition can be, for example, a β-glucan composition having an aggregate number less than about 6 under specified conditions. In one embodiment, the high molecular weight fraction which is separated from the starting composition is substantially in a triple helix aggregate conformation under physiological conditions. The high molecular weight portion can be any portion of the starting composition, as long as it has a greater average molecular weight than that of the starting composition. In one embodiment, the isolated portion represents about 60% or less, by weight, of the starting composition. The fraction of the starting composition isolated will depend upon the dispersion of molecular weights within the starting composition and the aggregate number desired and can be readily determined by one of skill in the art.

The high molecular weight portion can be separated from the starting composition using a variety of techniques. In a preferred embodiment, the high molecular weight portion is separated from the remainder of the starting composition using gel permeation chromatography (GPC). In this embodiment, the high molecular weight portion is separated from the starting composition by a method comprising the steps of (1) directing a β-glucan composition through a gel permeation chromatography column, and (2) collecting a high molecular weight fraction or a high molecular weight portion of a fraction of the starting composition.

In one embodiment, the starting β-glucan composition is separated into two or more fractions by GPC. In this case, the faster eluting fraction is a high molecular weight portion of the starting composition and all or a part of this fraction can be collected. In another embodiment, the starting β-glucan composition elutes as a single fraction or two or more overlapping fractions. In this case, the leading edge of the fraction or overlapping fractions can be collected.

The “leading edge” of a fraction eluting from a chromatography column is the portion of the fraction which elutes first. For example, if the fraction elutes in a given volume of eluent, the first 10 to 50% by volume of the fraction can be collected. The amount of the β-glucan fraction to be collected depends upon the nature of the original β-glucan composition, for example, the distribution of molecular weights and conformations, and the chromatography conditions, such as the type of GPC column employed, the eluent and the flow rate. Optimization of these parameters is within the ordinary level of skill in the art. β-Glucan molecules having higher aggregate numbers are expected to elute first. Therefore, if the portion collected has an aggregate number under physiological conditions which is lower than desired, the original β-glucan composition can be fractionated again, and a smaller leading edge portion can be collected to obtain a β-glucan composition having a larger aggregate number under physiological conditions. Preferably, the parameters are optimized using an analytical scale GPC column.

A suitable β-glucan composition having an aggregate number at physiological temperature of less than about 6 is a PGG-glucan composition previously described in U.S. Pat. No. 5,622,939. Preparative scale GPC can be performed to fractionate such a composition. For example, if the β-glucan composition elutes from the GPC column as a single band, the earlier-eluting, or leading edge, portion of the elution band can be collected to yield a PGG-glucan composition having an aggregate number greater than about 6. Such a β-glucan composition will have an increased triple helix aggregate conformer population at physiological temperature and pH compared to the original preparation.

The present invention also provides a method of preparing a soluble β-glucan composition having an aggregate number lower than that of a starting soluble β-glucan composition. The method comprises separating a low molecular weight portion from a starting soluble β-glucan composition. The low molecular weight portion is enriched in a single triple helix and/or single helix conformation compared to the starting composition. In one embodiment, the low molecular weight portion which is separated from the starting composition is substantially in a single triple helix conformation under physiological conditions. The low molecular weight portion can be any portion of the starting composition, as long as it has a lower average molecular weight than that of the starting composition. In one embodiment, the isolated portion represents about 60% or less, by weight, of the starting composition. The fraction of the starting composition separated will depend upon the dispersion of molecular weights within the starting composition and the aggregate number desired and can be readily determined by one of skill in the art.

The low molecular weight portion can be separated from the starting composition using a variety of techniques. In a preferred embodiment, the low molecular weight portion is separated from the remainder of the starting composition using gel permeation chromatography. In this embodiment, the high molecular weight portion is separated from the starting composition by a method comprising the steps of (1) directing a β-glucan composition through a gel permeation chromatography column, and (2) collecting a low molecular weight fraction or a low molecular weight portion of a fraction of the starting composition.

In one embodiment, the starting β-glucan composition is separated into two or more fractions by GPC. In this case, the more slowly eluting fraction is a low molecular weight portion of the starting composition and all or a part of this fraction can be collected. In another embodiment, the starting β-glucan composition elutes as a single fraction or two or more overlapping fractions. In this case, the trailing edge of the fraction or overlapping fractions can be collected.

The “trailing edge” of a fraction eluted from a chromatography column is that portion of the fraction which elutes last. For example, if the fraction elutes in a given volume of eluent, the last 10 to 50% of the fraction can be collected. The amount of the β-glucan fraction to be collected depends upon the nature of the original β-glucan composition, for example, the distribution of molecular weights and conformations, and the chromatography conditions, such as the type of gel permeation chromatography column employed, the eluent and the flow rate. Optimization of these parameters is within the ordinary level of skill in the art. β-Glucan molecules which adopt a single triple helix conformation under physiological conditions are expected to elute last. Therefore, if the portion collected has an aggregate number under physiological conditions which is greater than desired, the original β-glucan composition can be fractionated again, and a smaller trailing edge portion can be collected to obtain a β-glucan composition having a smaller aggregate number under physiological conditions. Preferably, the parameters are optimized using an analytical scale GPC column.

In a further embodiment, the present invention provides a method of forming a β-glucan composition comprising β-glucan chains which are in a triple helix aggregate conformation. The method comprises the steps of (1) reacting a highly branched β-glucan under conditions sufficient to remove at least a portion of the branches to form a debranched β-glucan and (2) maintaining the debranched β-glucan under conditions sufficient for formation of a triple helix aggregate form.

The highly branched β-glucan is a β-glucan which is substantially more branched than PGG-glucan, for example, a β-glucan which is too highly branched to form triple helix aggregates. For example, the highly branched β-glucan can be at least about 25% branched. In a preferred embodiment, the branches are joined to the main chain via β(1,6)-glycosidic bonds. Suitable examples of highly branched β-glucans of this type include scleroglucan, which is about 30-33% branched, schizophyllan, lentinan, cinerean, grifolan and pestalotan. The highly branched β-glucan can be debranched by cleaving a portion of the bonds joining the branches to the main polymer chain. For example, when the branches are joined to the main polymer chain by β(1,6)-glycosidic bonds, the β(1,6)-glycosidic bonds can be hydrolyzed under conditions which leave the main polymer chain substantially intact. For example, hydrolysis of the β(1,6)-glycodsidic bonds can be catalyzed by an enzyme which preferentially cleaves β(1,6)-glycosidic bonds β(1,3)-glycosidic bonds. Such enzymes of this type include hydrolases which are specific for or preferentially cleave β(1,6)-glycosidic bonds, for example, endoglycosidases, such as β(1,6)-glycosidases (Sasaki et al., Carbohydrate Res. 47: 99-104 (1976)).

The highly branched β-glucan can also be debranched using chemical methods. A preferred chemical debranching method is the Smith degradation (Whistler et al., Methods Carbohydrate Chem. 1:47-50 (1962)). In this method the β-glucan is treated for about 3 days in the dark with a limiting amount of NaIO₄, based on the extent of debranching desired. The reaction is next quenched with ethylene glycol and dialyzed. The reaction mixture is then treated with excess NaBH₄, then quenched with acetic acid and dialyzed. The reaction mixture is then heated for about 3 hours at 80° C. with 0.2 M trifluoroacetic acid. The reaction mixture is then dialyzed and concentrated.

The debranching reaction is performed under conditions suitable for forming a β-glucan composition which is sufficiently debranched to permit triple helix aggregate formation. For example, in one embodiment, the extent of branching of the debranched β-glucan is less than about 10%. In a preferred embodiment, the debranched β-glucan is branched to substantially the same extent as PGG-glucan (about 7%).

Indications

The soluble β-glucan compositions of the present invention have utility as safe, effective, therapeutic and/or prophylactic agents, either alone or as adjuvants, to enhance the immune response in humans and animals. An individual skilled in the medical arts will be able to determine the length of time during which the composition is administered and the dosage, depending on the physical condition of the patient and the disease or disorder being treated. As stated above, the composition may also be used as a preventative treatment to pre-initiate the normal metabolic defenses which the body mobilizes against infections. β-glucans produced by the present method preferably selectively activate only those components that are responsible for the initial response to infection, without stimulating or priming the immune system to release certain biochemical mediators (e.g., IL-1, TNF, IL-8 and GM-CSF) that can cause adverse side effects. As such, the present soluble glucan composition can be used to prevent or treat infectious diseases in malnourished patients, patients undergoing surgery and bone marrow transplants, patients undergoing chemotherapy or radiotherapy, neutropenic patients, HIV-infected patients, trauma patients, burn patients, patients with chronic or resistant infections such as those resulting from myelodysplastic syndrome, and the elderly, all of who may have weakened immune systems. An immunocompromised individual is generally defined as a person who exhibits an attenuated or reduced ability to mount a normal cellular or humoral defense to challenge by infectious agents, e.g., viruses, bacteria, fungi and protozoa. A protein malnourished individual is generally defined as a person who has a serum albumin level of less than about 3.2 grams per deciliter (g/dl) and/or unintentional weight loss of greater than 10% of usual body weight.

More particularly, the method of the invention can be used to therapeutically or prophylactically treat animals or humans who are at a heightened risk of infection due to imminent surgery, injury, illness, radiation or chemotherapy, or other condition which deleteriously affects the immune system. The method is useful to treat patients who have a disease or disorder which causes the normal metabolic immune response to be reduced or depressed, such as HIV infection (AIDS). For example, the method can be used to pre-initiate the metabolic immune response in patients who are undergoing chemotherapy or radiation therapy, or who are at a heightened risk for developing secondary infections or post-operative complications because of a disease, disorder or treatment resulting in a reduced ability to mobilize the body's normal metabolic responses to infection. Treatment with the soluble glucans has been shown to be particularly effective in mobilizing the host's normal immune defenses, thereby engendering a measure of protection from infection in the treated host.

β-glucan compositions can be used for the prevention and treatment of infections caused by a broad spectrum of bacterial, fungal, viral and protozoan pathogens. The prophylactic administration of β-glucan to a person undergoing surgery, either preoperatively, intraoperatively and/or post-operatively, will reduce the incidence and severity of post-operative infections in both normal and high-risk patients. For example, in patients undergoing surgical procedures that are classified as contaminated or potentially contaminated (e.g., gastrointestinal surgery, hysterectomy, cesarean section, transurethral prostatectomy) and in patients in whom infection at the operative site would present a serious risk (e.g., prosthetic arthroplasty, cardiovascular surgery), concurrent initial therapy with an appropriate antibacterial agent and the present β-glucan preparation will reduce the incidence and severity of infectious complications.

In patients who are immunosuppressed, not only by disease (e.g., cancer, AIDS) but by courses of chemotherapy and/or radiotherapy, the prophylactic administration of the β-glucan will reduce the incidence of infections caused by a broad spectrum of opportunistic pathogens including many unusual bacteria, fungi and viruses. Therapy using β-glucan has demonstrated a significant radio-protective effect with its ability to enhance and prolong macrophage function and regeneration and, as a result enhance resistance to microbial invasion and infection.

In high risk patients (e.g., over age 65, diabetics, patients having cancer, malnutrition, renal disease, emphysema, dehydration, restricted mobility, etc.) hospitalization frequently is associated with a high incidence of serious nosocomial infection. Treatment with β-glucan may be started empirically before catheterization, use of respirators, drainage tubes, intensive care units, prolonged hospitalizations, etc. to help prevent the infections that are commonly associated with these procedures. Concurrent therapy with antimicrobial agents and the β-glucan is indicated for the treatment of chronic, severe, refractory, complex and difficult to treat infections.

Another particular use of the compositions of this invention is for the treatment of myelodysplastic syndrome (MDS). MDS, frequently referred to as preleukemia syndrome, is a group of clonal hematopoietic stem cell disorders characterized by abnormal bone marrow differentiation and maturation leading to peripheral cytopenia with high probability of eventual leukemic conversion. Recurrent infection, hemorrhaging and terminal infection resulting in death typically accompany MDS. Thus, in order to reduce the severity of the disease and the frequency of infection, compositions comprising modified glucan can be chronically administered to a patient diagnosed as having MDS according to the methods of this invention, in order to specifically increase the infection fighting activity of the patient's white blood cells. Other bone marrow disorders, such as aplastic anemia (a condition of quantitatively reduced and defective hematopoiesis) can be treated to reduce infection and hemorrhage that are associated with this disease state.

The β-glucan compositions of the invention are also of use in methods of inducing or enhancing mobilization of peripheral blood precursor cells, elevating circulating levels of peripheral blood precursor cells and enhancing or facilitating hematopoietic reconstitution or engraftment in mammals, including humans. Peripheral blood precursor cells include stem cells and early progenitor cells which, although more differentiated than stem cells, have a greater potential for proliferation than stem cells. These methods comprise administering to the mammal an effective amount of a β-glucan composition of the present invention. Such methods are of use, for example, in the treatment of patients undergoing cytoreductive therapy, such as chemotherapy or radiation therapy.

β-glucan produced by the present method enhances the non-specific defenses of mammalian mononuclear cells and significantly increases their ability to respond to an infectious challenge. The unique property of β-glucan macrophage activation is that it does not result in increased body temperature (i.e., fever) as has been reported with many non-specific stimulants of those defenses. This critical advantage of β-glucan may lie in the natural profile of responses it mediates in white blood cells. It has been shown that the neutral soluble β-glucan of the present invention selectively activates immune responses but does not directly stimulate or prime cytokine (e.g., IL-1 and TNF) release from mononuclear cells, thus distinguishing the present β-glucan from other glucan preparations (e.g., lentinan, kresein) and immunostimulants.

In addition, it has been demonstrated herein that the β-glucan preparation of the present invention possesses an unexpected platelet stimulating property. Although it was known that glucans have the ability to stimulate white blood cell hematopoiesis, the disclosed platelet stimulating property had not been reported or anticipated. This property can be exploited in a therapeutic regimen for use as an adjuvant in parallel with radiation or chemotherapy treatment. Radiation and chemotherapy are known to result in neutropenia (reduced polymorphonuclear (PMN) leukocyte cell count) and thrombocytopenia (reduced platelet count). At present, these conditions are treated by the administration of colony stimulating factors such as GM-CSF and G-CSF. Such factors are effective in overcoming neutropenia, but fail to impact upon thrombocytopenia. Thus, the platelet stimulating property of β-glucans can be used, for example, as a therapeutic agent to prevent or minimize the development of thrombocytopenia which limits the dose of the radiation or chemotherapeutic agent which is used to treat cancer.

Administration

The present composition is generally administered to an animal or a human in an amount sufficient to produce immune system enhancement. The β-glucan portion of the combination composition of the invention can be administered parenterally by injection, e.g., subcutaneously, intravenously, intramuscularly, intraperitoneally, topically, orally or intranasaly. The β-glucans can be administered as a clear solution having a concentration of from about 1 mg/ml to about 5 mg/ml. The solvent can be a physiologically acceptable aqueous medium, such as water, saline, PBS or a 5% dextrose solution. The amount necessary to induce immune system enhancement will vary on an individual basis and be based at least in part on consideration of the individual's size, the severity of the symptoms and the results sought.

The β-glucan portion of the composition of the invention is generally administered to an animal or a human in an amount sufficient to produce immune system enhancement. The mode of administration of the β-glucan can be oral, enteral, parenteral, intravenous, subcutaneous, intraperitoneal, intramuscular, topical or intranasal. The form in which the β-glucan will be administered (e.g., powder, tablet, capsule, solution, emulsion) will depend on the route by which it is administered. The quantity of β-glucan to be administered will be determined on an individual basis, and will be based at least in part on consideration of the severity of infection or injury in the patient, the patient's condition or overall health, the patient's weight and the time available before surgery, chemotherapy or other high-risk treatment. In general, a single dose will preferably contain approximately 0.01 to approximately 10 mg of modified glucan per kilogram of body weight, preferably from about 0.1 to 2.5 mg/kg and more preferably from about 0.25 to about 2 mg/kg. The dosage for topical application will depend upon the particular wound to be treated, the degree of infection and severity of the wound. A typical dosage for wounds will be from about 0.001 mg/ml to about 2 mg/ml, and preferably from about 0.01 to about 0.5 mg/ml.

In general, the composition of the present invention can be administered to an individual periodically as necessary to stimulate the individual's immune response. An individual skilled in the medical arts will be able to determine the length of time during which the composition is administered and the dosage, depending on the physical condition of the patient and the disease or disorder being treated. As stated above, the composition may also be used as a preventative treatment to pre-initiate the normal metabolic defenses which the body mobilizes against infections.

The β-glucan portion of the compositions administered in the method of the present invention can optionally include other components, in addition to the neutral soluble β-glucans. The other components that can be included in a particular composition are determined primarily by the manner in which the composition is to be administered. For example, a composition to be administered orally in tablet form can include, in addition to neutral soluble β-glucan, a filler (e.g., lactose), a binder (e.g., carboxymethyl cellulose, gum arabic, gelatin), an adjuvant, a flavoring agent, a coloring agent and a coating material (e.g., wax or plasticizer). A β-glucan portion of the composition to be administered in liquid form can include neutral soluble β-glucan and, optionally, an emulsifying agent, a flavoring agent and/or a coloring agent. A β-glucan portion of the composition for parenteral administration can be mixed, dissolved or emulsified in water, sterile saline, phosphate buffered saline (PBS), dextrose or other biologically acceptable carrier. A composition for topical administration can be formulated into a gel, ointment, lotion, cream or other form in which the composition is capable of coating the site to be treated, e.g., wound site.

The β-glucan portion of the composition of the invention can also be administered topically to a wound site to stimulate and enhance wound healing and repair. Wounds due to ulcers, acne, viral infections, fungal infections or periodontal disease, among others, can be treated according to the methods of this invention to accelerate the healing process. Alternatively, the neutral soluble β-glucan can be injected into the wound or afflicted area. In addition to wound repair, the composition of the invention can be used to treat infection associated therewith or the causative agents that result in the wound. β-glucan portion of the composition of the invention for topical administration can be formulated into a gel, ointment, lotion, cream or other form in which the composition is capable of coating the site to be treated, e.g., wound site. The dosage for topical application will depend upon the particular wound to be treated, the degree of infection and severity of the wound. A typical dosage for wounds will be from about 0.01 mg/ml to about 2 mg/ml, and preferably from about 0.01 to about 0.5 mg/ml of β-glucan.

Anti-EGF Receptor Antagonists

Anti-EGF receptor antagonists include Cetuximab (ERBITUX™) disclosed in U.S. Pat. No. 6,217,866, incorporated herein by reference. Cetuximab is a recombinant, mouse/human chimeric monoclonal antibody which binds to the extracellular domain of the human EGF receptor, blocking the binding of EGF to its receptor, thereby inhibiting growth in cells which express EGF receptor, such as tumor cells. U.S. Pat. No. 6,217,866 discloses two monoclonal antibodies numbered 96 and 108 from cell lines ATCC HB 9763 and 9764, respectively. Both antibodies are also contemplated as part of the combination composition of the invention.

These antibodies, were made through the injection of Balb/c mice with CH 71 cells, which are Chinese Hamster Ovary (CHO) cells which have been transfected with a plasmid containing a truncated form of EGF receptor cDNA, which had most of the DNA encoding the intracellular portion of the EGF receptor deleted. The mice were immunized with this truncated receptor on days 0, 13 and 32, and the spleen cells of the two best responding mice were fused with NS1 myeloma cells according to the methods of Kohler and Milstein, Eur. J. Immuno., Vol. 6, 511-519 (1976). The fusion product was diluted in hypoxanthineazaserine (HA) selection medium (G. Buttin et al. Current Topics in Microbiology and Immunology, Vol. 81, 27-36 (1978)) and grown on 96 well plates.

The presence of the antibodies was detected by radioimmunoassay. Cells expressing the EGF receptor on their surface and control cells without EGF receptor expression were grown in separate wells on 96 well plates. Hybridomas which generated antibodies which specifically bound to the EGF receptor expressing cells and not the control cells were cloned by limiting their dilution and tested by their ability to immunoprecipitate ³⁵S methionine or ³²P labeled EGF receptor. It was shown that the “108” monoclonal antibody had antitumor activity against human oral epidermoid carcinoma cells in vitro and prolonged the life spans of nude mice injected with these cells in vivo. It was also shown that the “96” antibody inhibited cell growth of human breast cancer cells while not affecting the growth of human mammary epithelial cells in vitro.

The heavy and light variable regions of the “96” and “108” antibodies were cloned and recombinantly expressed. These proteins were refolded and competed for binding with the respective monoclonal antibodies that they were formed from, as described in U.S. Pat. No. 6,217,866. Thus, the composition of the invention also encompasses any antibody antibody fragment which is able to specifically bind with the EGF receptor, thereby blocking EGF signaling through its receptor.

Antibody Structure

The basic whole antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable domain of about 100 to 110 or more amino acids primarily responsible for antigen recognition. The carboxy-terminal portion of each chain defines a constant region primarily responsible for effector function. Human light chains are classified as kappa and lambda light chains. Human heavy chains are classified as mu, delta, gamma, alpha, or epsilon, and define the antibody's isotype as IgM, IgG, IgA, and IgE, respectively. Within light and heavy chains, the variable and constant regions are joined by a “3” region of about 12 or more amino acids, with the heavy chain also including a “D” region of about 10 more amino acids. See generally, Fundamental Immunology Ch. 7 (Paul, W., ed., 2d ed. Raven Press, N.Y. (1989)) (incorporated by reference in its entirety for all purposes). The variable regions of each light/heavy chain pair form the antibody binding site.

The variable domains all exhibit the same general structure of relatively conserved framework regions (FR) joined by three hyper variable regions, also called complementarity determining regions or CDRs. The CDRs from the heavy and light chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each region is in accordance with the definitions of Kabat, Sequences of Proteins of Immunological Interest (National Institutes of Health, Bethesda, Md. (1987 and 1991)), or Chothia & Lesk, J. Mol. Biol. 196:901-917 (1987); Chothia et al., Nature 342:878-883 (1989).

Human Antibodies and Humanization of Antibodies

Embodiments of the invention described herein contemplate and encompass human antibodies. Human antibodies avoid certain of the problems associated with antibodies that possess murine or rat variable and/or constant regions. The presence of such murine or rat derived proteins can lead to the rapid clearance of the antibodies or can lead to the generation of an immune response against the antibody by a mammal other than a rodent.

The ability to clone and reconstruct megabase-sized human loci in YACs and to introduce them into the mouse germline provides a powerful approach to elucidating the functional components of very large or crudely mapped loci as well as generating useful models of human disease. An important practical application of such a strategy is the “humanization” of the mouse humoral immune system. Introduction of human immunoglobulin (Ig) loci into mice in which the endogenous Ig genes have been inactivated offers the opportunity to develop human antibodies in the mouse. Fully human antibodies are expected to minimize the immunogenic and allergic responses intrinsic to mouse or mouse-derivatized monoclonal antibodies and thus to increase the efficacy and safety of the antibodies administered to humans. The use of fully human antibodies can be expected to provide a substantial advantage in the treatment of chronic and recurring human diseases, such as inflammation, autoimmunity, and cancer, which require repeated antibody administrations.

One approach toward this goal was to engineer mouse strains deficient in mouse antibody production with large fragments of the human Ig loci in anticipation that such mice would produce a large repertoire of human antibodies in the absence of mouse antibodies. This general strategy was demonstrated in connection with the generation of the first XenoMouse® strains as published in 1994. See Green et al., Nature Genetics 7:13-21 (1994).

Alternative approaches have utilized a “minilocus” approach, in which an exogenous Ig locus is mimicked through the inclusion of pieces (individual genes) from the Ig locus. Thus, one or more V_{H} genes, one or more D_{H} genes, one or more J_{H} genes, a mu constant region, and a second constant region (preferably a gamma constant region) are formed into a construct for insertion into an animal. This approach is described in U.S. Pat. No. 5,545,807 to Surani et al. and U.S. Pat. Nos. 5,545,806, 5,625,825, 5,625,126, 5,633,425, 5,661,016, 5,770,429, 5,789,650, 5,814,318, 5,877,397, 5,874,299, and 6,255,458 each to Lonberg and Kay, U.S. Pat. Nos. 5,591,669 and 6,023,010 to Krimpenfort and Berns, U.S. Pat. Nos. 5,612,205, 5,721,367, and 5,789,215 to Berns et al., and U.S. Pat. No. 5,643,763 to Choi and Dunn, and GenPharm International U.S. patent application Ser. No. 07/574,748, filed Aug. 29, 1990, 07/575,962, filed Aug. 31, 1990, 07/810,279, filed Dec. 17, 1991, 07/853,408, filed Mar. 18, 1992, 07/904,068, filed Jun. 23, 1992, 07/990,860, filed Dec. 16, 1992, 08/053,131, filed Apr. 26, 1993, 08/096,762, filed Jul. 22, 1993, 08/155,301, filed Nov. 18, 1993, 08/161,739, filed Dec. 3, 1993, 08/165,699, filed Dec. 10, 1993, 08/209,741, filed Mar. 9, 1994, the disclosures of which are hereby incorporated by reference. See also European Patent No. 0 546 073 B1, International Patent Application Nos. WO 92/03918, WO 92/22645, WO 92/22647, WO 92/22670, WO 93/12227, WO 94/00569, WO 94/25585, WO 96/14436, WO 97/13852, and WO 98/24884 and U.S. Pat. No. 5,981,175, the disclosures of which are hereby incorporated by reference in their entirety. See further Taylor et al., 1992, Chen et al., 1993, Tuaillon et al., 1993, Choi et al., 1993, Lonberg et al., (1994), Taylor et al., (1994), and Tuaillon et al., (1995), Fishwild et al., (1996), the disclosures of which are hereby incorporated by reference in their entirety.

While chimeric antibodies have a human constant region and a murine variable region, it is expected that certain human anti-chimeric antibody (HACA) responses will be observed, particularly in chronic or multi-dose utilizations of the antibody.

Humanization and Display Technologies

Antibodies with reduced immunogenicity can be generated using humanization and library display techniques. It will be appreciated that antibodies can be humanized or primatized using techniques well known in the art. See e.g., Winter and Harris, Immunol Today 14:43-46 (1993) and Wright et al., Crit, Reviews in Immunol. 12:125-168 (1992). The antibody of interest can be engineered by recombinant DNA techniques to substitute the CH1, CH2, CH3, hinge domains, and/or the framework domain with the corresponding human sequence (see WO 92/02190 and U.S. Pat. Nos. 5,530,101, 5,585,089, 5,693,761, 5,693,792, 5,714,350, and 5,777,085). Also, the use of Ig cDNA for construction of chimeric immunoglobulin genes is known in the art (Liu et al., P.N.A.S. 84:3439 (1987) and J. Immunol. 139:3521 (1987)). mRNA is isolated from a hybridoma or other cell producing the antibody and used to produce cDNA. The cDNA of interest can be amplified by the polymerase chain reaction using specific primers (U.S. Pat. Nos. 4,683,195 and 4,683,202). Alternatively, an expression library is made and screened to isolate the sequence of interest encoding the variable region of the antibody is then fused to human constant region sequences. The sequences of human constant regions genes can be found in Kabat et al., “Sequences of Proteins of Immunological Interest,” N.I.H. publication no. 91-3242 (1991). Human C region genes are readily available from known clones. The choice of isotype will be guided by the desired effector functions, such as complement fixation, or activity in antibody-dependent cellular cytotoxicity. Preferred isotypes are IgG1, IgG2 and IgG4. Either of the human light chain constant regions, kappa or lambda, can be used. The chimeric, humanized antibody is then expressed by conventional methods. Expression vectors include plasmids, retroviruses, YACs, EBV derived episomes, and the like.

Antibody fragments, such as Fv, F(ab′)₂ and Fab can be prepared by cleavage of the intact protein, e.g., by protease or chemical cleavage. Alternatively, a truncated gene is designed. For example, a chimeric gene encoding a portion of the F(ab′)₂ fragment would include DNA sequences encoding the CH1 domain and hinge region of the H chain, followed by a translational stop codon to yield the truncated molecule.

Consensus sequences of H and L J regions can be used to design oligonucleotides for use as primers to introduce useful restriction sites into the J region for subsequent linkage of V region segments to human C region segments. C region cDNA can be modified by site directed mutagenesis to place a restriction site at the analogous position in the human sequence.

Expression vectors include plasmids, retroviruses, YACs, EBV derived episomes, and the like. A convenient vector is one that encodes a functionally complete human CH or CL immunoglobulin sequence, with appropriate restriction sites engineered so that any VH or VL sequence can be easily inserted and expressed. In such vectors, splicing usually occurs between the splice donor site in the inserted J region and the splice acceptor site preceding the human C region, and also at the splice regions that occur within the human CH exons. Polyadenylation and transcription termination occur at native chromosomal sites downstream of the coding regions. The resulting chimeric antibody can be joined to any strong promoter, including retroviral LTRs, e.g., SV-40 early promoter, (Okayama et al., Mol. Cell. Bio. 3:280 (1983)), Rous sarcoma virus LTR (Gorman et al., P.N.A.S. 79:6777 (1982)), and moloney murine leukemia virus LTR (Grosschedl et al., Cell 41:885 (1985)). Also, as will be appreciated, native Ig promoters and the like can be used.

Further, human antibodies or antibodies from other species can be generated through display-type technologies, including, without limitation, phage display, retroviral display, ribosomal display, and other techniques, using techniques well known in the art and the resulting molecules can be subjected to additional maturation, such as affinity maturation, as such techniques are well known in the art. Wright and Harris, supra., Hanes and Plucthau, PNAS USA 94:4937-4942 (1997) (ribosomal display), Parmley and Smith, Gene 73:305-318 (1988) (phage display), Scott, TIBS 17:241-245 (1992), Cwirla et al., PNAS USA 87:6378-6382 (1990), Russel et al., Nucl. Acids Res. 21:1081-1085 (1993), Hoganboom et al., Immunol. Reviews 130:43-68 (1992), Chiswell and McCafferty, TIBTECH 10:80-84 (1992), and U.S. Pat. No. 5,733,743. If display technologies are utilized to produce antibodies that are not human, such antibodies can be humanized as described above.

Other Anti-EGF Receptor Antagonists

The anti-EGF receptor antagonists used in the composition of the invention are not limited to Cetuximab, or even antibodies themselves. Monoclonal or polyclonal antibodies, antibody fragments or other proteins or small molecules are also contemplated by the invention. The one property these molecules must share is the ability to specifically bind to the EGF receptor so as to block the interaction of EGF with the receptor, thereby preventing mitogenic events associated with EGF.

Specific molecules that are contemplated for use in the composition of the invention include the following. Monoclonal or polyclonal antibodies which bind to the EGF receptor from various species including rat, mouse, horse, cow, goat, sheep, pig and rabbit are contemplated for use in the composition of the invention. Also, chimeric antibodies, other than Cetuximab, produced from a human antibody and monoclonal antibodies made in any of the above mentioned animals are contemplated for use in the composition of the invention. Antibody fragments, especially variable regions from antibodies which bind to EGF-receptor are contemplated for use in the composition of the invention. Also, EGF mutants which, while still able to bind to the EGF receptor, do not cause mitogenic signaling through the receptor and block antigenic signaling of wild-type EGF are contemplated for use in the composition of the invention. Small molecules which bind to EGF receptor and block EGF signaling through the receptor are also contemplated for use in the composition of the invention. Further, soluble EGF receptor fragments, for example, encompassing the extracellular domain of the EGF receptor, which are able to bind EGF thereby preventing EGF from binding to cell expressed wild-type EGF receptor are also contemplated for use in the composition of the invention.

Indications

EGF receptor antagonists are used to treat cancer. For example, Cetuximab has been FDA approved to treat colon cancer. However, EGF receptor antagonists have also been found effective against breast and oral epidermoid carcinoma cells. Other cancers which are treated by EGF receptor antagonists are ovarian, breast, prostate, colon, pancreatic, multiple myeloma, malignant melanoma and non-melanoma skin cancers.

EGF receptor antagonists are suitable for the reduction of cancer symptoms. These cancer symptoms include blood in the urine, pain or burning upon urination, frequent urination, cloudy urine, pain in the bone or swelling around the affected site, fractures in bones, weakness, fatigue, weight loss, repeated infections, nausea, vomiting, constipation, problems with urination, weakness or numbness in the legs, bumps and bruises that persist, dizziness, drowsiness, abnormal eye movements or changes in vision, weakness, loss of feeling in arms or legs or difficulties in walking, fits or convulsions, changes in personality, memory or speech, headaches that tend to be worse in the morning and ease during the day, that may be accompanied by nausea or vomiting, a lump or thickening of the breast, discharge from the nipple, change in the skin of the breast, a feeling of heat, or enlarged lymph nodes under the arm, rectal bleeding (red blood in stools or black stools), abdominal cramps, constipation alternating with diarrhea, weight loss, loss of appetite, weakness, pallid complexion, dull ache or pain in the back or side, lump in kidney area, sometimes accompanied by high blood pressure or abnormality in red blood cell count, weakness, paleness, fever and flu-like symptoms, bruising and prolonged bleeding, enlarged lymph nodes, spleen, liver, pain in bones and joints, frequent infections, weight loss, night sweats, wheezing, persistent cough for months, blood-streaked sputum, persistent ache in chest, congestion in lungs, enlarged lymph nodes in the neck, change in mole or other bump on the skin, including bleeding or change in size, shape, color, or texture, painless swelling in the lymph nodes in the neck, underarm, or groin, persistent fever, feeling of fatigue, unexplained weight loss, itchy skin and rashes, small lumps in skin, bone pain, swelling in the abdomen, liver or spleen enlargement, a lump in the mouth, ulceration of the lip, tongue or inside of the mouth that does not heal within a couple of weeks, dentures that no longer fit well, oral pain, bleeding, foul breath, loose teeth, changes in speech, abdominal swelling, abnormal vaginal bleeding, digestive discomfort, upper abdominal pain, unexplained weight loss, pain near the center of the back, intolerance of fatty foods, yellowing of the skin, abdominal masses, enlargement of liver and spleen, urination difficulties due to blockage of the urethra, bladder retains urine, creating frequent feelings of urgency to urinate, especially at night, bladder not emptying completely, burning or painful urination, bloody urine, tenderness over the bladder, dull ache in the pelvis or back, indigestion or heartburn, discomfort or pain in the abdomen, nausea and vomiting, diarrhea or constipation, bloating after meals, loss of appetite, weakness and fatigue, bleeding—vomiting blood or blood in the stool, abnormal vaginal bleeding, a watery bloody discharge in postmenopausal women, a painful urination, pain during intercourse, and pain in pelvic area.

It has been found that Cetuximab is useful in treating cancer in mammals. Cetuximab and other EGF receptor antagonists or antibody fragments are also combined with other anti-cancer drugs for the treatment of cancer, for example, doxorubicin, cisplatin and irinotecan. Anti-cancer drugs are also contemplated as part of the combination composition of the invention. Other anti-cancer drugs, for example, include taxanes, nitrogen mustards, ethylenimine derivatives, alkyl sulfonates, nitrosoureas, triazenes; folic acid analogs, pyrimidine analogs, purine analogs, vinca alkaloids, antibiotics, enzymes, platinum coordination complexes, substituted urea, methyl hydrazine derivatives, adrenocortical suppressants, or antagonists. More specifically, the chemotherapeutic agents may be one or more agents chosen from the non-limiting group of steroids, progestins, estrogens, antiestrogens, or androgens. Even more specifically, the chemotherapy agents may be azaribine, bleomycin, bryostatin-1, busulfan, carmustine, chlorambucil, CPT-11, cyclophosphamide, cytarabine, dacarbazine, dactinomycin, daunorubicin, dexamethasone, diethylstilbestrol, doxorubicin, ethinyl estradiol, etoposide, fluorouracil, fluoxymesterone, gemcitabine, hydroxyprogesterone caproate, hydroxyurea, L-asparaginase, leucovorin, lomustine, mechlorethamine, medroprogesterone acetate, megestrol acetate, melphalan, mercaptopurine, methotrexate, methotrexate, mithramycin, mitomycin, mitotane, phenyl butyrate, prednisone, procarbazine, semustine streptozocin, tamoxifen, taxanes, taxol, testosterone propionate, thalidomide, thioguanine, thiotepa, uracil mustard, vinblastine, or vincristine.

Administration

The recommended dose of Cetuximab is 400 mg/m² as an initial loading dose (first infusion) administered as a 120-minute IV infusion (maximum infusion rate 5 mL/min) administered intravenously. The recommended weekly maintenance dose (all other infusions) is 250 mg/m² infused over 60 minutes (maximum infusion rate 5 mL/min). Following a 2-hour infusion of 400 mg/m² of Cetuximab, the maximum mean serum concentration (Cmax) was 184 μg/mL (range: 92-327 μg/mL) and the mean elimination half-life was 97 hours (range 41-213 hours). A 1-hour infusion of 250 mg/m² produced a mean Cmax of 140 μg/mL (range 120-170 μg/mL). Following the recommended dose regimen (400 mg/m² initial dose/250 mg/m² weekly dose), Cetuximab concentrations reached steady-state levels by the third weekly infusion with mean peak and trough concentrations across studies ranging from 168 to 235 and 41 to 85 μg/mL, respectively. The mean half-life was 114 hours (range 75-188 hours). Other administration methods are contemplated as described below.

Combination Composition

The composition of the invention is a combination of β-glucan and EGF receptor antagonist. Any of the above described β-glucans can be combined with any of the above described EGF receptor antagonists. The combination composition allows lower dosages of β-glucan or EGF receptor antagonist or both to be administered to an animal. Further, the combination composition leads to additive and synergistic effects.

Synergy is defined as the interaction of two or more agents so that their combined effect is greater than the sum of their individual effects. For example, if the effect of drug A alone in treating a disease is 25%, and the effect of drug B alone in treating a disease is 25%, but when the two drugs are combined the effect in treating the disease is 75%, the effect of A and B is synergistic.

For example, β glucans have a wide range of use in enhancing immune response in humans. EGF receptor antagonists like Cetuximab are effective in treating cancer, and are often co-administered with chemotherapeutic agents or radiation treatments. Combination therapy with β-glucans would reduce side-effects associated with these co-administered cancer drugs, thus allowing higher doses of cancer drugs or lower doses of Cetuximab or both. Further, β-glucans are useful for treating myelodysplastic syndrome (MDS), which has a high possibility of conversion to leukemia. Thus, β-glucans co-administered with Cetuximab would act to prevent the leukemic conversion, but also treat leukemia if it did occur. Therefore, the combination of β-glucans with EGF receptor antagonists in one agent affords synergistic protection not provided by either agent alone.

Additivity is defined as the interaction of two or more agents so that their combined effect is greater than the sum of their individual effects. For example, if the effect of drug A alone in treating a disease is 25%, and the effect of drug B alone in treating a disease is 25%, but when the two drugs are combined the effect in treating the disease is greater than 25%, the effect of A and B is additive.

An improvement in the drug therapeutic regimen can be described as the interaction of two or more agents so that their combined effect reduces the incidence of adverse event (AE) of either or both agents used in co-therapy. This reduction in the incidence of adverse effects can be a result of, e.g., administration of lower dosages of either or both agent used in the co-therapy. For example, if the effect of Drug A alone is 25% and has an adverse event incidence of 45% at labeled dose; and the effect of Drug B alone is 25% and has an adverse event incidence of 30% at labeled dose, but when the two drugs are combined at lower than labeled doses of each, if the overall effect is 35%. and the adverse incidence rate is 20%, there is an improvement in the drug therapeutic regimen.

In one embodiment, methods of treating a proliferative disorder, such as cancer, are disclosed, wherein a β-glucan and an EGF receptor antagonist are administered to a subject having a proliferative disorder such as cancer, such that the cancer is treated or at least partially alleviated. The β-glucan and EGF receptor antagonist may be administered as part of a pharmaceutical composition, or as part of a combination therapy. In another embodiment, a patient is diagnosed, e.g., to determine if treatment is necessary, whereupon a combination therapy in accordance with the invention is administered to treat the patient. The amount of β-glucan and EGF receptor antagonist is typically effective to reduce symptoms and to enable an observation of a reduction in symptoms.

Combination therapies of a β-glucan, e.g., BETAFECTIN™ and pharmaceutically acceptable salts and esters thereof; and EGF receptor antagonist such as Cetuximab are synergistically effective and are effective in treating a proliferative disorder such as cancer.

In another embodiment, methods of treating a immune dysfunction, such as infection in an immunocompromised patient, are disclosed, wherein a β-glucan and an EGF receptor antagonist are administered to a subject having a immune dysfunction such as infection in an immunocompromised patient, such that the infection in an immunocompromised patient is treated or at least partially alleviated. The β-glucan and EGF receptor antagonist may be administered as part of a pharmaceutical composition, or as part of a combination therapy. In another embodiment, a patient is diagnosed, e.g., to determine if treatment is necessary, whereupon a combination therapy in accordance with the invention is administered to treat the patient. The amount of β-glucan and EGF receptor antagonist is typically effective to reduce symptoms and to enable an observation of a reduction in symptoms.

Combination therapies of a β-glucan, e.g., BETAFECTIN™ and pharmaceutically acceptable salts and esters thereof; and EGF receptor antagonist such as Cetuximab are synergistically effective and are effective in treating a immune dysfunction such as infection in an immunocompromised patient.

Reduced Side Effects/Other Benefits

Accordingly, the combination of the invention allows the β-glucan and the EGF receptor antagonist to be administered in a combination that improves efficacy and avoids undesirable side effects of both drugs. For example, side effects include airway obstruction, including bronchospasm, stridor, or hoarseness; urticaria, hypotension, interstitial lung disease, inflammation, renal pathology, acneform rash, skin drying and fissuring, fever, sepsis, kidney failure, pulmonary embolus, dehydration, diarrhea, abdominal pain, vomiting, and inflammatory and infectious sequelae, for example plepharitis, cheilitis, cellulites or cyst. Side-effects associated with the administration of EGF receptor antagonist may be lessened in severity and frequency through co-administration of β-glucan. Similarly, side effects associated with the use of β-glucan may be reduced in severity and frequency through controlled release methods as well.

Dosages Taken Together

The β-glucans used in combination therapies of the invention are administered at a dosage of generally, from about 0.01 to about 10 mg/kg/day. More preferably the dose of β-glucan is from about 0.1 to about 2.5 mg/kg/day. BETAFECTIN™ is particularly preferred.

The EGF receptor antagonists used in combination therapies of the invention are administered at a dosage of generally, from about 100 to about 800 mg/m²/week. More preferably the dose of β-glucan is from about 250 to about 400 mg/m²/week. In a preferred embodiment from about 200 to about 400 mg/m² of Cetuximab is administered in a first infusion, followed by a weekly infusion of from about 125 to about 150 mg/m² of Cetuximab.

When taken together, the β-glucan and EGF receptor antagonist may be administered in a single weekly infusion. Doses of β-glucan administered per week range from about 0.07 to about 70 mg/kg/week. The β-glucan and EGF receptor antagonist may also be administered daily, wherein the daily dose for the EGF receptor antagonist is from about 14 to about 114 mg/m²/week.

Schedule of Administration

As noted above, combination therapies of a β-glucan and an EGF receptor antagonist are part of the invention. The combination therapies of the invention are administered in any suitable fashion to obtain the desired treatment of a proliferative disorder (e.g., cancer) or immune dysfunction (e.g. infection) in the patient. One way in which this is achieved is to prescribe a regimen of β-glucan so as to “pre-treat” the patient to obtain the effects of the β-glucan (e.g. a slowing of disease progression and neuroprotection), then follow that up with the EGF receptor antagonist as part of a specific treatment regimen, e.g., a standard administration of Cetuximab, e.g., intravenously, to provide the benefit of the co-action of the therapeutic agents. Combination therapies of the invention include this sequential administration, as well as administration of these therapeutic agents, or at least two of the therapeutic agents, in a substantially simultaneous manner. Substantially simultaneous administration can be accomplished, for example, by administering to the subject a single infusion having a fixed ratio of a β-glucan and, EGF receptor antagonist, or in multiple, single injections. The components of the combination therapies, as noted above, can be administered by the same route or by different routes. For example, a β-glucan is administered orally, while the EGF receptor antagonists is administered intravenously; or all therapeutic agents may be administered by intravenous injection. The sequence in which the therapeutic agents are administered is not believed to be critical.

Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence in which the therapeutic agents are administered is not narrowly critical.

“Combination therapy” also can embrace the administration of the therapeutic agents as described above in further combination with other biologically active ingredients and non-drug therapies (e.g., surgery or radiation treatment.) Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

Thus, the compounds of the invention and the other pharmacologically active agent may be administered to a patient simultaneously, sequentially or in combination. If administered sequentially, the time between administrations generally varies from 0.1 to about 48 hours. It will be appreciated that when using a combination of the invention, the compound of the invention and the other pharmacologically active agent may be in the same pharmaceutically acceptable carrier and therefore administered simultaneously. They may be in separate pharmaceutical carriers such as conventional oral dosage forms which are taken simultaneously. The term “combination” further refers to the case where the compounds are provided in separate dosage forms and are administered sequentially.

Other pharmacological agents which are administered with the combination composition of the invention include anti-cancer drugs, for example, doxorubicin, cisplatin and irinotecan. Other anti-cancer drugs include taxanes, nitrogen mustards, ethylenimine derivatives, alkyl sulfonates, nitrosoureas, triazenes; folic acid analogs, pyrimidine analogs, purine analogs, vinca alkaloids, antibiotics, enzymes, platinum coordination complexes, substituted urea, methyl hydrazine derivatives, adrenocortical suppressants, or antagonists. More specifically, the chemotherapeutic agents may be one or more agents chosen from the non-limiting group of steroids, progestins, estrogens, antiestrogens, or androgens. Even more specifically, the chemotherapy agents may be azaribine, bleomycin, bryostatin-1, busulfan, carmustine, chlorambucil, CPT-11, cyclophosphamide, cytarabine, dacarbazine, dactinomycin, daunorubicin, dexamethasone, diethylstilbestrol, doxorubicin, ethinyl estradiol, etoposide, fluorouracil, fluoxymesterone, gemcitabine, hydroxyprogesterone caproate, hydroxyurea, L-asparaginase, leucovorin, lomustine, mechlorethamine, medroprogesterone acetate, megestrol acetate, melphalan, mercaptopurine, methotrexate, methotrexate, mithramycin, mitomycin, mitotane, phenyl butyrate, prednisone, procarbazine, semustine streptozocin, tamoxifen, taxanes, taxol, testosterone propionate, thalidomide, thioguanine, thiotepa, uracil mustard, vinblastine, or vincristine.

A combination therapy for a proliferative disorder includes BETAFECTIN™ and Cetuximab. In another embodiment, a combination therapy for a proliferative disorder includes BETAFECTIN™, Cetuximab and irinotecan. In another embodiment, a combination therapy for a proliferative disorder includes BETAFECTIN™, Cetuximab and cisplatin. In another embodiment, a combination therapy for a proliferative disorder includes BETAFECTIN™, Cetuximab and doxorubicin.

In another embodiment, a combination therapy for a immune dysfunction includes BETAFECTIN™ and Cetuximab.

The present invention provides a more effective method of treatment for proliferative disorders, and pharmaceutical compositions for treating proliferative disorders which may be used in such methods. In an embodiment, the invention relates to methods for treating proliferative disorders through the administration of one or more β-glucans in combination with EGF receptor antagonists and, optionally other treatments, such as anti-cancer drugs and treatments.

The present invention provides a more effective method of treatment for immune dysfunctions, and pharmaceutical compositions for treating immune dysfunctions which may be used in such methods. In an embodiment, the invention relates to methods for treating immune dysfunctions through the administration of one or more β-glucans in combination with EGF receptor antagonists.

The beneficial effect of the combination composition of the invention includes, but is not limited to, pharmacokinetic or pharmacodynamic co-action resulting from the combination of therapeutic agents. In one embodiment, the co-action of the therapeutic agents is additive. In another embodiment, the co-action of the therapeutic agents is synergistic. In another embodiment, the co-action of the therapeutic agents improves the therapeutic regimen of one or both of the agents.

The invention further relates to kits for treating patients having a proliferative disorder, such as cancer, comprising a therapeutically effective dose of at least one EGF receptor antagonist (e.g., Cetuximab), and a β-glucan, either in the same or separate packaging, and instructions for its use. The kit optionally further comprises a therapeutically effective dose of an anti-cancer drug such as irinotecan.

The invention further relates to kits for treating patients having a immune dysfunction, such as infection, comprising a therapeutically effective dose of at least one EGF receptor antagonist (e.g., Cetuximab), and a β-glucan, either in the same or separate packaging, and instructions for its use.

The present invention is suitable for the reduction of proliferative disorder symptoms. These symptoms include blood in the urine, pain or burning upon urination, frequent urination, cloudy urine, pain in the bone or swelling around the affected site, fractures in bones, weakness, fatigue, weight loss, repeated infections, nausea, vomiting, constipation, problems with urination, weakness or numbness in the legs, bumps and bruises that persist, dizziness, drowsiness, abnormal eye movements or changes in vision, weakness, loss of feeling in arms or legs or difficulties in walking, fits or convulsions, changes in personality, memory or speech, headaches that tend to be worse in the morning and ease during the day, that may be accompanied by nausea or vomiting, a lump or thickening of the breast, discharge from the nipple, change in the skin of the breast, a feeling of heat, or enlarged lymph nodes under the arm, rectal bleeding (red blood in stools or black stools), abdominal cramps, constipation alternating with diarrhea, weight loss, loss of appetite, weakness, pallid complexion, dull ache or pain in the back or side, lump in kidney area, sometimes accompanied by high blood pressure or abnormality in red blood cell count, weakness, paleness, fever and flu-like symptoms, bruising and prolonged bleeding, enlarged lymph nodes, spleen, liver, pain in bones and joints, frequent infections, weight loss, night sweats, wheezing, persistent cough for months, blood-streaked sputum, persistent ache in chest, congestion in lungs, enlarged lymph nodes in the neck, change in mole or other bump on the skin, including bleeding or change in size, shape, color, or texture, painless swelling in the lymph nodes in the neck, underarm, or groin, persistent fever, feeling of fatigue, unexplained weight loss, itchy skin and rashes, small lumps in skin, bone pain, swelling in the abdomen, liver or spleen enlargement, a lump in the mouth, ulceration of the lip, tongue or inside of the mouth that does not heal within a couple of weeks, dentures that no longer fit well, oral pain, bleeding, foul breath, loose teeth, changes in speech, abdominal swelling, abnormal vaginal bleeding, digestive discomfort, upper abdominal pain, unexplained weight loss, pain near the center of the back, intolerance of fatty foods, yellowing of the skin, abdominal masses, enlargement of liver and spleen, urination difficulties due to blockage of the urethra, bladder retains urine, creating frequent feelings of urgency to urinate, especially at night, bladder not emptying completely, burning or painful urination, bloody urine, tenderness over the bladder, dull ache in the pelvis or back, indigestion or heartburn, discomfort or pain in the abdomen, nausea and vomiting, diarrhea or constipation, bloating after meals, loss of appetite, weakness and fatigue, bleeding—vomiting blood or blood in the stool, abnormal vaginal bleeding, a watery bloody discharge in postmenopausal women, a painful urination, pain during intercourse, and pain in pelvic area.

Preferably, treatment should continue as long as proliferative disorder symptoms are suspected or observed.

To evaluate whether a patient is benefiting from the (treatment), one would examine the patient's symptoms in a quantitative way, by decrease in the frequency of relapses, or increase in the time to sustained progression. In a successful treatment, the patient status will have improved, measurement number or frequency of relapses will have decreased, or the time to sustained progression will have increased.

As for every drug, the dosage is an important part of the success of the treatment and the health of the patient. In every case, in the specified range, the physician has to determine the best dosage for a given patient, according to gender, age, weight, height, pathological state and other parameters.

The pharmaceutical compositions of the present invention contain a therapeutically effective amount of the active agents. The amount of the compound will depend on the patient being treated. The patient's weight, severity of illness, manner of administration and judgment of the prescribing physician should be taken into account in deciding the proper amount. The determination of a therapeutically effective amount of an β-glucan or EGF receptor antagonist is well within the capabilities of one with skill in the art.

In some cases, it may be necessary to use dosages outside of the ranges stated in pharmaceutical packaging insert to treat a patient. Those cases will be apparent to the prescribing physician. Where it is necessary, a physician will also know how and when to interrupt, adjust or terminate treatment in conjunction with a response of a particular patient.

Formulation (Separately or Together) and Administration

The compounds of the present invention are administered separately or co-formulated in a suitable co-formulated dosage form. Compounds, including those used in combination therapies are administered to a patient in the form of a pharmaceutically acceptable salt or in a pharmaceutical composition. A compound that is administered in a pharmaceutical composition is mixed with a suitable carrier or excipient such that a therapeutically effective amount is present in the composition. The term “therapeutically effective amount” refers to an amount of the compound that is necessary to achieve a desired endpoint (e.g., decreasing symptoms associated with cancer).

A variety of preparations can be used to formulate pharmaceutical compositions containing the β-glucans and EGF receptor antagonists. Techniques for formulation and administration may be found in “Remington: The Science and Practice of Pharmacy, Twentieth Edition,” Lippincott Williams & Wilkins, Philadelphia, Pa. Tablets, capsules, pills, powders, granules, dragees, gels, slurries, ointments, solutions suppositories, injections, inhalants and aerosols are examples of such formulations. The formulations can be administered in either a local or systemic manner or in a depot or sustained release fashion. Administration of the composition can be performed in a variety of ways. The compositions and combination therapies of the invention may be administered in combination with a variety of pharmaceutical excipients, including stabilizing agents, carriers and/or encapsulation formulations as described herein.

The preparation of pharmaceutical or pharmacological compositions will be known to those of skill in the art in light of the present disclosure. Typically, such compositions may be prepared as injectables, either as liquid solutions or suspensions; solid forms suitable for solution in, or suspension in, liquid prior to injection; as tablets or other solids for oral administration; as time release capsules; or in any other form currently used, including creams, lotions, mouthwashes, inhalants and the like.

For human administration, preparations should meet sterility, pyrogenicity, general safety and purity standards as required by the FDA.

Administration of compounds alone or in combination therapies may be, e.g., subcutaneous, intramuscular or intravenous injection, or any other suitable route of administration. A particularly convenient frequency for the administration of the compounds of the invention is once a day.

Upon formulation, therapeutics will be administered in a manner compatible with the dosage formulation, and in such amount as is pharmacologically effective. The formulations are easily administered in a variety of dosage forms, such as the injectable solutions described, but drug release capsules and the like can also be employed. In this context, the quantity of active ingredient and volume of composition to be administered depends on the host animal to be treated. Precise amounts of active compound required for administration depend on the judgment of the practitioner and are peculiar to each individual.

A minimal volume of a composition required to disperse the active compounds is typically utilized. Suitable regimes for administration are also variable, but would be typified by initially administering the compound and monitoring the results and then giving further controlled doses at further intervals.

A carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Suitable preservatives for use in solution include benzalkonium chloride, benzethonium chloride, chlorobutanol, thimerosal and the like. Suitable buffers include boric acid, sodium and potassium bicarbonate, sodium and potassium borates, sodium and potassium carbonate, sodium acetate, sodium biphosphate and the like, in amounts sufficient to maintain the pH at between about pH 6 and pH 8, and preferably, between about pH 7 and pH 7.5. Suitable tonicity agents are dextran 40, dextran 70, dextrose, glycerin, potassium chloride, propylene glycol, sodium chloride, and the like, such that the sodium chloride equivalent of the ophthalmic solution is in the range 0.9 plus or minus 0.2%. Suitable antioxidants and stabilizers include sodium bisulfite, sodium metabisulfite, sodium thiosulfite, thiourea and the like. Suitable wetting and clarifying agents include polysorbate 80, polysorbate 20, poloxamer 282 and tyloxapol. Suitable viscosity-increasing agents include dextran 40, dextran 70, gelatin, glycerin, hydroxyethylcellulose, hydroxmethylpropylcellulose, lanolin, methylcellulose, petrolatum, polyethylene glycol, polyvinyl alcohol, polyvinylpyrrolidone, carboxymethylcellulose and the like.

The compounds and combination therapies of the invention can be formulated by dissolving, suspending or emulsifying in an aqueous or nonaqueous solvent. Vegetable (e.g., sesame oil, peanut oil) or similar oils, synthetic aliphatic acid glycerides, esters of higher aliphatic acids and propylene glycol are examples of nonaqueous solvents. Aqueous solutions such as Hank's solution, Ringer's solution or physiological saline buffer can also be used. In all cases the form must be sterile and must be fluid to the extent that easy syringability exists. It must be stable under the conditions of manufacture and storage and must be preserved against the contaminating action of microorganisms, such as bacteria and fungi.

Solutions of active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The preparation of more, or highly, concentrated solutions for subcutaneous or intramuscular injection is also contemplated. In this regard, the use of DMSO as solvent is preferred as this will result in extremely rapid penetration, delivering high concentrations of the active compound(s) or agent(s) to a small area.

Where one or both active ingredients of the combination therapy is given orally, it can be formulated through combination with pharmaceutically acceptable carriers that are well known in the art. The carriers enable the compound to be formulated, for example, as a tablet, pill, capsule, solution, suspension, sustained release formulation; powder, liquid or gel for oral ingestion by the patient. Oral use formulations can be obtained in a variety of ways, including mixing the compound with a solid excipient, optionally grinding the resulting mixture, adding suitable auxiliaries and processing the granule mixture. The following list includes examples of excipients that can be used in an oral formulation: sugars such as lactose, sucrose, mannitol or sorbitol; cellulose preparations such as maize starch, wheat starch, potato starch, gelatin, gum tragacanth, methyl cellulose, hydroxypropylmethylcellulose, sodium carboxymethylcellulose and polyvinylpyrrolidone (PVP). Oral formulations include such normally employed excipients as, for example, pharmaceutical grades of mannitol, lactose, starch, magnesium stearate, sodium saccharine, cellulose, magnesium carbonate and the like.

In certain defined embodiments, oral pharmaceutical compositions will comprise an inert diluent or assimilable edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, buccal tables, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 75% of the weight of the unit, or preferably between 25-60%. The amount of active compounds in such therapeutically useful compositions is such that a suitable dosage will be obtained.

The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid cannier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent methyl and propylparabensas preservatives, a dye and flavoring, such as cherry or orange flavor.

Additional formulations suitable for other modes of administration include suppositories. For suppositories, traditional binders and carriers may include, for example, polyalkylene glycols or triglycerides; such suppositories may be formed from mixtures containing the active ingredient in the range of 0.5% to 10%, preferably 1%-2%.

The subject treated by the methods of the invention is a mammal, more preferably a human. The following properties or applications of these methods will essentially be described for humans although they may also be applied to non-human mammals, e.g., apes, monkeys, dogs, mice, etc. The invention therefore can also be used in a veterinarian context.

In one embodiment the combination compositions disclosed herein can also be formulated as liposomes. Liposomes containing the compositions of the invention are prepared by methods known in the art, such as described in Epstein et al., Proc. Natl. Acad. Sci. USA, 82: 3688 (1985); Hwang et al., Proc. Natl. Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Compositions of the present invention can be conjugated to the liposomes as described in Martin et al., J. Biol. Chem., 257: 286-288 (1982) via a disulfide-interchange reaction.

The invention is further illustrated by the following Examples.

### EXAMPLES

### Example 1

### Efficacy of Cetuximab in Treatment of Cancer

The efficacy and safety of Cetuximab alone and in combination with irinotecan was studied in a randomized, controlled trial (329 patients) and in combination with irinotecan in an open-label, single-arm trial (138 patients). Cetuximab was further evaluated as a single agent in a third clinical trial (57 patients). Safety data from 111 patients treated with single agent Cetuximab was also evaluated. All trials studied patients with EGFR-expressing metastatic colorectal cancer, whose disease had progressed after receiving an irinotecan-containing regimen.

Randomized, Controlled Trial

A multicenter, randomized, controlled clinical trial was conducted in 329 patients randomized to receive either Cetuximab plus irinotecan (218 patients) or Cetuximab monotherapy (111 patients). In both arms of the study, Cetuximab was administered as a 400 mg/m² initial dose, followed by 250 mg/m² weekly until disease progression or unacceptable toxicity. All patients received a 20-mg test dose on Day 1. In the Cetuximab plus irinotecan arm, irinotecan was added to Cetuximab using the same dose and schedule for irinotecan as the patient had previously failed. Acceptable irinotecan schedules were 350 mg/m² every 3 weeks, 180 mg/m² every 2 weeks, or 125 mg/m² weekly times four doses every 6 weeks. An Independent Radiographic Review Committee (IRC), blinded to the treatment arms, assessed both the progression on prior irinotecan and the response to protocol treatment for all patients. Of the 329 randomized patients, 206 (63%) were male. The median age was 59 years (range 26-84), and the majority was Caucasian (323, 98%). Fifty-eight percent of patients had colon cancer and 40% rectal cancer. Approximately two-thirds (63%) of patients had previously failed oxaliplatin treatment. The efficacy of Cetuximab plus irinotecan or Cetuximab monotherapy was evaluated in all randomized patients. Analyses were also conducted in two pre-specified subpopulations: irinotecan refractory and irinotecan and oxaliplatin failures. The irinotecan refractory population was defined as randomized patients who had received at least two cycles of irinotecan-based chemotherapy prior to treatment with Cetuximab, and had independent confirmation of disease progression within 30 days of completion of the last cycle of irinotecan-based chemotherapy.

The irinotecan and oxaliplatin failure population was defined as irinotecan refractory patients who had previously been treated with and failed an oxaliplatin-containing regimen.

The objective response rates (ORR) in these populations are presented in Table 1. Objective response rates are the sum of the complete and partial response rates. A complete response would be the disappearance of all detectable tumor from the patient. A partial response would be a decrease in tumor size of greater than 50%, but the tumor would still be detectable in the patient.

TABLE 1
Objective Response Rates per Independent Review

| | Cetuximab + Irinotecan | | Cetuximab Monotherapy | | Difference (95% CIₐ) | |
|---|---|---|---|---|---|---|
| Populations | n | ORR (%) | n | ORR (%) | % | p-value CMH^{_{b}} |
| All Patients | 218 | 22.9 | 111 | 10.8 | 12.1 | 0.007 |
| | | | | | (4.1-20.2) | |
| Irinotecan- | 80 | 23.8 | 44 | 11.4 | 12.4 | 0.09 |
| Oxaliplatin | | | | | (−0.8, 25.6) | |
| Failure | | | | | | |
| Irinotecan | 132 | 25.8 | 69 | 14.5 | 11.3 | 0.07 |
| Refractory | | | | | (0.1-22.4) | |

ₐ95% confidence interval for the difference in objective response rates.
_{b}Cochran-Mantel-Haenszel test.

The median duration of response in the overall population was 5.7 months in the combination arm and 4.2 months in the monotherapy arm. Compared with patients randomized to Cetuximab alone, patients randomized to Cetuximab and irinotecan experienced a significantly longer median time to disease progression (see Table 2).

TABLE 2
Time to Progression per Independent Review

| | Cetuximab + | Cetuximab | | |
|---|---|---|---|---|
| | Irinotecan | Monotherapy | Hazard Ratio | Log-rank |
| Populations | (median) | (median) | (95% CIₐ) | p-value |
| All Patients | 4.1 mo | 1.5 mo | 0.54 (0.42-0.71) | <0.001 |
| Irinotecan- | 2.9 mo | 1.5 mo | 0.48 (0.31-0.72) | <0.001 |
| Oxaliplatin | | | | |
| Failure | | | | |
| Irinotecan | 4.0 mo | 1.5 mo | 0.52 (0.37-0.73) | <0.001 |
| Refractory | | | | |

ₐHazard ratio of Cetuximab + irinotecan: Cetuximab monotherapy with 95% confidence interval.

Single-Arm Trials

Cetuximab, in combination with irinotecan, was studied in a single-arm, multicenter, open-label clinical trial in 138 patients with EGFR-expressing metastatic colorectal cancer who had progressed following an irinotecan containing regimen. Patients received a 20-mg test dose of Cetuximab on day 1, followed by a 400-mg/m² initial dose, and 250 mg/m² weekly until disease progression or unacceptable toxicity. Patients received the same dose and schedule for irinotecan as the patient had previously failed. Acceptable irinotecan schedules were 350 mg/m² every 3 weeks or 125 mg/m² weekly times four doses every 6 weeks. Of 138 patients enrolled, 74 patients had documented progression to irinotecan as determined by an IRC. The overall response rate was 15% for the overall population and 12% for the irinotecan failure population. The median durations of response were 6.5 and 6.7 months, respectively.

### Example 2

### Infection Model

A sepsis model was developed in rats to characterize the efficacy of PGG glucans in protecting an immunologically intact host against serious infections, such as those which commonly occur following abdominal surgery. The rat model for intra-abdominal sepsis has been well described in the scientific literature (Onderdonk et al., 1974, Infect. Immun., 10:1256, 1259).

Groups of rats received β-glucan (100 μg/0.2 ml) or saline control (0.2 ml) intramuscularly 24 hours and 4 hours prior to infectious challenge. A defined polymicrobic infectious challenge (cecal inoculum) was placed into a gelatin capsule which was then surgically implanted into the peritoneal cavity of anesthetized rats through an anterior midline incision. The early peritonitis from this experimentally induced infection was associated with the presence of gram-negative organisms within the blood and peritoneal cavity culminating in mortality. The cecal inoculum contained an array of facultative species, such E. coli, as well as other obligate anaerobes (Streptococcus sp., Bacteroides sp., Clostridium perfringens, Clostridium ramosum, Peptostreptococcus magnus and productus, Proteus mirabilis). The animals were observed four times per day for the first 48 h and twice per day thereafter. The results are reported in Table 3.

TABLE 3
Effect of β-Glucan on Mortality in a Rat Model for Intra-abdominal Sepsis

| Group | Mortality (%) | P vs. Saline |
|---|---|---|
| Saline | 12/20 (60) | |
| β-glucan | 2/10 (10) | <0.01 |

These results demonstrate that β-glucan which does not induce IL-1 and TNF protects rats from lethal bacterial challenge.

### Example 3

### Administration of Neutral Soluble Glucan to Humans

A randomized, double-blind, placebo-controlled clinical trial was conducted on healthy males to evaluate the safety of neutral soluble glucan (2.25 mg/kg) injected by intravenous infusion compared to a placebo control. No adverse effects were observed. There was also no observed elevation in IL-1, TNF, IL-6, IL-8 and GM-CSF. Single intraveneous administration of neutral soluble glucan resulted in increases in monocytes and neutrophils and in the killing activity of these cells proving that neutral soluble glucan retains the desirable immunological activities in humans. See Tables 4, 5 and 6 below. However, no changes occurred in serum IL-1 and TNF and none of the patients experienced fever or inflammatory reactions. The results are consistent with the in vitro data reported in the earlier examples.

TABLE 4
Change In Absolute Neutrophil Counts (×1000/μl) After
Neutral Soluble Glucan Administration

| Dose Level | | B | Hour 8 | Hour 12 | Hour 24 |
|---|---|---|---|---|---|
| Saline | Mean | 4.06 | 4.34 | 4.31 | 3.43 |
| | STD | 2.12 | 1.53 | 1.16 | 1.46 |
| | N | 6 | 6 | 6 | 6 |
| 2.5 mg/kg | Mean | 4.11 | 11.29* | 8.18 | 5.32 |
| Neutral | STD | 1.15 | 4.39 | 3.80 | 1.75 |
| Soluble | N | 6 | 6 | 6 | 6 |
| Glucan | | | | | |

B = Baseline measurement
*p < 0.01 with respect to baseline

TABLE 5
Change in Monocyte Counts (×1000/μl) After
Soluble Neutral Glucan Administration

| Dose Level | | B | Hour 8 | Hour 12 | Hour 24 |
|---|---|---|---|---|---|
| Saline | Mean | 0.33 | 0.44 | 0.59 | 0.33 |
| | STD | 0.09 | 0.10 | 0.22 | 0.12 |
| | N | 6 | 6 | 6 | 6 |
| 2.5 mg/kg | Mean | 0.24 | 0.63* | 0.67* | 0.31 |
| Neutral | STD | 0.10 | 0.24 | 0.32 | 0.15 |
| Soluble | N | 6 | 6 | 6 | 6 |
| Glucan | | | | | |

B = Baseline measurement
*p < 0.01 with respect to baseline

TABLE 6
Ex Vivo Microbicidal Activity of Normal Volunteers
Receiving Neutral Soluble Glucan Mean Change in % Killing¹

| Dose | | Hour | Hour | Hour | Day | Day | Day |
|---|---|---|---|---|---|---|---|
| Level | | 3 | 6 | 24 | 2 | 3 | 6 |
| Saline | | 0 | 0 | 0 | 0 | 0 | 0 |
| 2.5 mg/ | Mean | 42.86 | 32.33 | 20.90 | 48.96 | 39.22 | 31.17 |
| kg | | | | | | | |
| Neutral | N | 6 | 6 | 6 | 6 | 6 | 6 |
| Soluble | p- | 0.062 | 0.036 | 0.300 | 0.045 | 0.085 | 0.026 |
| Glucan | value | | | | | | |

¹Normalized with respect to the saline control

### Example 4

### Trials for Efficacy of Combination Compositions

In this example, qualified animal models for cancer are employed to examine the dose ranges of synergistic interaction of β-glucan and EGF-receptor antibodies.

Treatment with BETAFECTIN™ and Cetuximab (ERBITUX™)

Animal Models and Methods

Colony Inhibition Assay of KB Cells.

Human oral epidermoid carcinoma (KB) cells are seeded in petri dishes (50×15 mm², NUNC) at a concentration of 2×10² cells per dish. After 16 to 24 hours medium is replaced with a fresh one containing BETAFECTIN™, Cetuximab or combinations of the two all at varying concentrations. On the sixth day cultures are fed with fresh medium containing the same ingredients. On the 15th day the cultures are washed with PBS, fixed with 4% v/v formaldehyde in PBS for 15 min. and stained with hematoxylin. Number of formed colonies (25 cells) is then determined.

Antitumoral Activity of BETAFECTIN™ and Cetuximab Combination Compositions.

KB cells (2×10⁶) are injected subcutaneously into nude mice, followed by either one or several intravenal injections of BETAFECTIN™, Cetuximab, combinations of the two, or saline control at varying doses, starting one day after tumor cell injection. Tumor parameters are measured twice a week with a caliper and its volume was calculated according to the formula: Tumor volume (mm³)=length×width×height. In order to validate volume measurements, correlation between tumor volume and tumor weight at the day of animal killing is assessed.

Infection Model

A sepsis model was developed in rats to characterize the efficacy of PGG glucans in protecting an immunologically intact host against serious infections, such as those which commonly occur following abdominal surgery. The rat model for intra-abdominal sepsis has been well described in the scientific literature (Onderdonk et al., 1974, Infect. Immun., 10:1256, 1259).

Groups of rats received BETAFECTIN™, Cetuximab, combinations of the two or saline control intramuscularly 24 hours and 4 hours prior to infectious challenge. A defined polymicrobic infectious challenge (cecal inoculum) was placed into a gelatin capsule which was then surgically implanted into the peritoneal cavity of anesthetized rats through an anterior midline incision. The early peritonitis from this experimentally induced infection was associated with the presence of gram-negative organisms within the blood and peritoneal cavity culminating in mortality. The cecal inoculum contained an array of facultative species, such E. coli, as well as other obligate anaerobes (Streptococcus sp., Bacteroides sp., Clostridium perfringens, Clostridium ramosum, Peptostreptococcus magnus and productus, Proteus mirabilis). The animals were observed four times per day for the first 48 h and twice per day thereafter.

Study Assessment. Cells and animals are assessed for both arresting inflammation, cell growth, and tumor growth and increasing apoptosis.

### Example 5

### Methods of Producing Beta Glucans

To produce β-glucan, whole glucan particles are suspended in an acid solution under conditions sufficient to dissolve the acid-soluble glucan portion. For most glucans, an acid solution having a pH of from about 1 to about 5 and a temperature of from about 20 to about 100° C. is sufficient. Preferably, the acid used is an organic acid capable of dissolving the acid-soluble glucan portion. Acetic acid, at concentrations of from about 0.1 to about 5M or formic acid at concentrations of from about 50% to 98% (w/v) are useful for this purpose. The treatment is preferably carried out at about 90° C. The treatment time may vary from about 1 hour to about 20 hours depending on the acid concentration, temperature and source of whole glucan particles. For example, modified glucans having more β(1-6) branching than naturally-occurring, or wild-type glucans, require more stringent conditions, i.e., longer exposure times and higher temperatures. This acid-treatment step can be repeated under similar or variable conditions. In one embodiment of the present method, modified whole glucan particles from the strain, S. cerevisiae R4, which have a higher level of β(1-6) branching than naturally-occurring glucans, are used, and treatment is carried out twice: first with 0.5M acetic acid at 90° C. for 3 hours and second with 0.5M acetic acid at 90° C. for 20 hours.

The acid-insoluble glucan particles are then separated from the solution by an appropriate separation technique, for example, by centrifugation or filtration. The pH of the resulting slurry is adjusted with an alkaline compound such as sodium hydroxide, to a pH of about 7 to about 14. The slurry is then resuspended in hot alkali having a concentration and temperature sufficient to solubilize the glucan polymers. Alkaline compounds which can be used in this step include alkali-metal or alkali-earth metal hydroxides, such as sodium hydroxide or potassium hydroxide, having a concentration of from about 0.1 to about 10N. This step can be conducted at a temperature of from about 4° C. to about 121° C., preferably from about 20° C. to about 100° C. In one embodiment of the process, the conditions utilized are a 1N solution of sodium hydroxide at a temperature of about 80-100° C. and a contact time of approximately 1-2 hours. The resulting mixture contains solubilized glucan molecules and particulate glucan residue and generally has a dark brown color due to oxidation of contaminating proteins and sugars. The particulate residue is removed from the mixture by an appropriate separation technique, e.g., centrifugation and/or filtration.

The resulting solution contains soluble glucan molecules. This solution can, optionally, be concentrated to effect a 5 to 10 fold concentration of the retentate soluble glucan fraction to obtain a soluble glucan concentration in the range of about 1 to 5 mg/ml. This step can be carried out by an appropriate concentration technique, for example, by ultrafiltration, utilizing membranes with nominal molecular weight levels (NMWL) or cut-offs in the range of about 1,000 to 100,000 daltons. A membrane cut-off of about 10,000 daltons is particularly useful for this step.

The concentrated fraction obtained after this step is enriched in the soluble, biologically active glucan PGG. To obtain a pharmacologically acceptable solution, the glucan concentrate is further purified, for example, by diafiltration. In one embodiment of the present method, diafiltration is carried out using approximately 10 volumes of alkali in the range of about 0.2 to 0.4N. The preferred concentration of the soluble glucan after this step is from about 2 to about 5 mg/ml. The pH of the solution is adjusted in the range of about 7-9 with an acid, such as hydrochloric acid. Traces of proteinaceous material which may be present can be removed by contacting the resulting solution with a positively charged medium such as DEAE-cellulose, QAE-cellulose or Q-Sepharose. Proteinaceous material is detrimental to the quality of the glucan product, may produce a discoloration of the solution and aids in the formation of gel networks, thus limiting the solubility of the neutral glucan polymers. A clear solution is obtained after this step.

The highly purified, clear glucan solution can be further purified, for example, by diafiltration, using a pharmaceutically acceptable medium (e.g., sterile water for injection, phosphate-buffered saline (PBS), isotonic saline, dextrose) suitable for parenteral administration. The preferred membrane for this diafiltration step has a nominal molecular weight cut-off of about 10,000 daltons. The final concentration of the glucan solution is adjusted in the range of about 0.5 to 5 mg/ml. In accordance with pharmaceutical manufacturing standards for parenteral products, the solution can be terminally sterilized by filtration through a 0.22 μm filter. The soluble glucan preparation obtained by this process is sterile, non-antigenic, and essentially pyrogen-free, and can be stored at room temperature for extended periods of time without degradation.

A critical advantage of this method is that precipitation, drying or reconstitution of the soluble glucan polymer is not required at any point in the process. The resulting solution is substantially free of protein contamination, is non-antigenic, non-pyrogenic and is pharmaceutically acceptable for parenteral administration to animals and humans. However, if desired, the soluble glucan can be dried by an appropriate drying method, such as lyophilization, and stored in dry form. The dried glucan can be reconstituted prior to use by adding an alkali solution such as about 0.1-0.4N NaOH and reprocessed starting from the step immediately following the organic acid contact steps described above.

### Example 6

### Methods of Making Neutral Soluble Beta Glucans

In the present process, whole glucan particles are suspended in an acid solution under conditions sufficient to dissolve the acid-soluble glucan portion. For most glucans, an acid solution having a pH of from about 1 to about 5 and at a temperature of from about 20 to about 100° C. is sufficient. Preferably, the acid used is an organic acid capable of dissolving the acid-soluble glucan portion. Acetic acid, at concentrations of from about 0.1 to about 5M or formic acid at concentrations of from about 50% to 98% (w/v) are useful for this purpose. The treatment time may vary from about 10 minutes to about 20 hours depending on the acid concentration, temperature and source of whole glucan particles. For example, modified glucans having more β(1-6) branching than naturally-occurring, or wild-type glucans, require more stringent conditions, i.e., longer exposure times and higher temperatures. This acid-treatment step can be repeated under similar or variable conditions. One preferred processing method is described in the exemplification using glucan derived from S. cerevisiae strain R4 Ad. In another embodiment of the present method, whole glucan particles from the strain, S. cerevisiae R4, which have a higher level of β(1-6) branching than naturally occurring glucans, are used, and treatment is carried out with 90% (by wt.) formic acid at 20° C. for about 20 minutes and then at 85° C. for about 30 minutes.

The insoluble glucan particles are then separated from the solution by an appropriate separation technique, for example, by centrifugation or filtration. The pH of the resulting slurry is adjusted with an alkaline compound such as sodium hydroxide, to a pH of about 7 to about 14. The precipitate is collected by centrifugation and is boiled in purified water (e.g., USP) for three hours. The slurry is then resuspended in hot alkali having a concentration sufficient to solubilize the glucan polymers. Alkaline compounds which can be used in this step include alkali-metal or alkali-earth metal hydroxides, such as sodium hydroxide or potassium hydroxide, having a concentration of from about 0.01 to about 10N. This step can be conducted at a temperature of from about 4° C. to about 121° C., preferably from about 20° C. to about 100° C. In one embodiment of the process, the conditions utilized are a 1M solution of sodium hydroxide at a temperature of about 80-100° C. and a contact time of approximately 1-2 hours. The resulting mixture contains solubilized glucan molecules and particulate glucan residue and generally has a dark brown color due to oxidation of contaminating proteins and sugars. The particulate residue is removed from the mixture by an appropriate separation technique, e.g., centrifugation and/or filtration. In another embodiment of the process the acid-soluble glucans are precipitated after the preceding acid hydrolysis reaction by the addition of about 1.5 volumes of ethanol. The mixture is chilled to about 4° C. for two (2) hours and the resulting precipitate is collected by centrifugation or filtration and washed with water. The pellet is then resuspended in water, and stirred for three (3) to twelve (12) hours at a temperature between about 20° C. and 100° C. At this point the pH is adjusted to approximately 10 to 13 with a base such as sodium hydroxide.

The resulting solution contains dissociated soluble glucan molecules. This solution is now purified to remove traces of insoluble glucan and high molecular weight soluble glucans which can cause aggregation. This step can be carried out by an appropriate purification technique, for example, by ultrafiltration, utilizing membranes with nominal molecular weight levels (NMWL) or cut-offs in the range of about 1,000 to 100,000 daltons. It was discovered that in order to prevent gradual aggregation or precipitation of the glucan polymers the preferred membrane for this step has a nominal molecular weight cut-off of about 100,000 daltons. The soluble glucan is then further purified at alkaline pH to remove low molecular weight materials. This step can be carried out by an appropriate purification technique, for example, by ultrafiltration, utilizing membranes with nominal molecular weight levels or cut-offs in the range of 1,000 to 30,000 daltons.

The resulting dissociated soluble glucan is re-annealed under controlled conditions of time (e.g., from about 10 to about 120 minutes), temperature (e.g., from about 50 to about 70° C.) and pH. The pH of the solution is adjusted in the range of about 6-8 with an acid, such as hydrochloric acid. The purpose of this re-annealing step is to cause the soluble glucan to rearrange from a single helix conformation to a new ordered triple helical conformation. The re-annealed glucan solution is then size fractionated using 30,000-100,000 NMW and 150,000-500,000 NMW cut off membrane ultrafilters to selectively remove high and low molecular weight soluble glucans. Prior to sizing, the soluble glucans exist as a mixture of conformations including random coils, gel matrices or aggregates, triple helices and single helices. The objective of the sizing step is to obtain an enriched fraction for the re-annealed conformation of specific molecular weight. The order in which the ultrafilters are used is a matter of investigator preference and should result in the same desired product.

The concentrated fraction obtained after this step is enriched in the soluble, biologically active neutral soluble glucan. The glucan concentrate is further purified, for example, by diafiltration using a 10,000 dalton membrane. The preferred concentration of the soluble glucan after this step is from about 2 to about 10 mg/ml.

The neutralized solution is then further purified, for example, by diafiltration, using a pharmaceutically acceptable medium (e.g., sterile water for injection, phosphate-buffered saline (PBS), isotonic saline, dextrose) suitable for parenteral administration. The preferred membrane for this diafiltration step has a nominal molecular weight cutoff of about 10,000 daltons. The final concentration of the glucan solution is adjusted in the range of about 0.5 to 10 mg/ml. In accordance with pharmaceutical manufacturing standards for parenteral products, the solution can be terminally sterilized by filtration through a 0.22 μm filter. The neutral soluble glucan preparation obtained by this process is sterile, non-antigenic, and essentially pyrogen-free, and can be stored at room temperature (e.g., 15-30° C.) for extended periods of time without degradation. This process is unique in that it results in a neutral aqueous solution of (pH 4.5 to 7.0) immunologically active glucans which is suitable for parenteral administration.

The resulting solution is substantially free of protein contamination, is non-antigenic, non-pyrogenic and is pharmaceutically acceptable for parenteral administration to animals and humans. However, if desired, the soluble glucan can be dried by an appropriate drying method, such as lyophilization, and stored in dry form.

### Other Embodiments

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. Other aspects, advantages, and modifications are considered to be within the scope of the following claims. The claims presented are representative of the inventions disclosed herein. Other, unclaimed inventions are also contemplated. Applicants reserve the right to pursue such inventions in later claims.

What is claimed is:

1. A composition comprising a β-glucan and an EGF receptor antagonist.

2. The composition of claim 1, wherein the β-glucan forms a triple helix.

3. The composition of claim 2, wherein the triple helix β-glucan forms a higher order aggregate.

4. The composition of claim 3, wherein the higher order aggregate has an aggregate number selected from the group consisting of 5, 6, 7, 8, 9, 10, 11, 12, 13, 14, 15, 16, 17, 18, 19 and 20.

5. The composition of claim 1, wherein the EGF receptor antagonist is an antibody.

6. The composition of claim 5, wherein the antibody is one of polyclonal, monoclonal or a combination thereof.

7. The composition of claim 6, wherein the monoclonal antibody is antibody 108 or antibody 96.

8. The composition of claim 5, wherein the antibody is Cetuximab.

9. The composition of claim 1, further comprising an anti-cancer drug.

10. The composition of claim 9, wherein the anti-cancer drug is a member of the group consisting of ironotecan, doxorubicin and cisplatin.

11. The composition of claim 1, wherein the composition is administered to a subject.

12. The composition of claim 11, wherein the subject is a mammal.

13. A kit comprising a therapeutic dose of a β-glucan and a therapeutic dose of an EGF receptor antagonist either in the same or separate packaging, and instructions for its use.

14. The kit of claim 13, wherein the β-glucan forms a triple helix.

15. The kit of claim 14, wherein the triple helix β-glucan forms a higher order aggregate.

16. The kit of claim 13, wherein the EGF receptor antagonist is an antibody.

17. The kit of claim 16, wherein the antibody is Cetuximab.

18. A pharmaceutical composition comprising a β-glucan and an EGF receptor antagonist in an effective amount to treat one of cancer, infection or both.

19. The pharmaceutical composition of claim 18, wherein the β-glucan is a triple helical β-glucan and the EGF receptor antagonist is Cetuximab.

20. The pharmaceutical composition of claim 19, further comprising an anti-cancer drug in an effective amount to treat cancer.
